(19) United States
(12) Patent Application Publication
(10) Pub. No.: US 2017/0233762 A1
(43) Pub. Date: Aug. 17, 2017

(54) SCAFFOLD RNAS

(71) Applicant: The Regents of the University of California, Oakland, CA (US)

(72) Inventors: Jesse Zalatan, San Francisco, CA (US); Wendell Lim, San Francisco, CA (US); Lei Qi, San Francisco, CA (US)

(21) Appl. No.: 15/514,892

(22) PCT Filed: Sep. 29, 2015
(86) PCT No.: PCT/US15/53034

§ 371 Date: Mar. 28, 2017

Related U.S. Application Data
(60) Provisional application No. 62/057,120, filed on Sep. 29, 2014.

(51) Int. Cl. C12N 15/85; C12N 15/113

(52) U.S. Cl. CPC C12N 15/85; C12N 15/113

(57) ABSTRACT

Scaffold RNAs are provided. Compositions and methods are also provided for making and using scaffold RNAs.

### CROSS-REFERENCES TO RELATED APPLICATIONS

This application claims priority to U.S. Provisional Application No. 62/057,120, filed on Sep. 29, 2014, the contents of which are hereby incorporated by reference in the entirety for all purposes.

### STATEMENT AS TO RIGHTS TO INVENTIONS MADE UNDER FEDERALLY SPONSORED RESEARCH AND DEVELOPMENT

This invention was made with government support under grants no. P50 GM081879, EY016546, R01 DA055040, R01 DA036858 and OD017887 awarded by the National Institutes of Health. The government has certain rights in the invention.

### BACKGROUND OF THE INVENTION

A hallmark of biological systems is their use of spatial organization to link functional effector molecules to their target sites. The ability to link functional effector molecules to their target sites in a controlled and specific manner can also be a useful tool for synthetic biology. For example, methods and compositions providing such linkage can be used for transcriptional regulation (e.g., activation or inhibition) of target genetic elements.

### BRIEF SUMMARY OF THE INVENTION

In a first aspect, the present invention provides a scaffold RNA (scRNA), wherein the scaffold RNA comprises: a nucleic acid binding region, the nucleic acid binding region having a length of between about 15 to about 30 nucleotides, wherein the nucleic acid binding region is complementary to a target nucleic acid; a 5′ scaffold region, wherein the 5′ scaffold region is 5′ of a 3′ scaffold region and specifically binds to at least one 5′ scaffold region binding polypeptide or small molecule; the 3′ scaffold region, wherein the 3′ scaffold region is 3′ of the 5′ scaffold region and specifically binds to at least one 3′ scaffold region binding polypeptide or small molecule; and a transcription termination sequence, wherein the scaffold RNA is configured to recruit 5′ and 3′ scaffold region binding polypeptides or small molecules to the target nucleic acid.

In some embodiments, the 5′ scaffold region comprises one, two, or more RNA hairpins. In some embodiments, the 3′ scaffold region comprises one, two, or more RNA hairpins. In some embodiments the 5′ scaffold region is 5′ of the binding region. In some embodiments, the 5′ scaffold region is 3′ of the binding region. In some embodiments, the small molecule has a molecular weight of less than about 5,000; less than about 1,000; or less than about 500 daltons.

In some embodiments, the binding of a small molecule or polypeptide to the 5′ scaffold region and/or the 3′ scaffold region mediates the activity of the scRNA. In some embodiments, the binding of a small molecule to the 5′ scaffold region and/or the 3′ scaffold region mediates the binding of a polypeptide to the 5′ scaffold region and/or the 3′ scaffold region. In some cases, the activity of the scRNA comprises transcriptional modulation, chromatin modification, or target genetic element binding.

In some embodiments, the 5′ scaffold region and/or the 3′ scaffold region is configured to bind a small guide RNA-mediated nuclease (e.g., Cas9, nickase Cas9, or dCas9), and the scaffold region configured to bind the small guide RNA-mediated nuclease is 3′ of the nucleic acid binding region. In some cases, the 5′ scaffold region and/or the 3′ scaffold region that is configured to bind a small guide RNA-mediated nuclease is encoded by a sequence comprising SEQ ID NO:1 or SEQ ID NO:13.

In some cases, the 5′ scaffold region and/or the 3′ scaffold region is configured to bind two or more polypeptides. The two or more polypeptides can each be structurally different or at least two of the two or more polypeptides can comprise the same polypeptide sequence. In some cases, at least two of the two or more polypeptides are monomers of a homodimer. In some cases, at least two of the two or more polypeptides are monomers of a heterodimer.

In some embodiments, the 5′ scaffold region and/or the 3′ scaffold region is configured to bind one or more, or two or more, polypeptides, wherein at least one of the polypeptides comprises a transcriptional modulator and an affinity domain having affinity for the 5′ scaffold region or the 3′ scaffold region. In some cases, the transcriptional modulator comprises a transcriptional activator. In some cases, the transcriptional activator is VP16 or VP64. In some cases, the transcriptional modulator comprises a transcriptional repressor. In some cases, the transcriptional repressor is a KRAB domain. In some cases, the transcriptional modulator comprises a chromatin modifier. In some cases, the chromatin modifier comprises an enzyme that methylates or demethylates DNA or histones, or an enzyme that acetylates or deacetylates histones.

In some embodiments, the 5′ scaffold region and/or the 3′ scaffold region each comprises an ms2, f6, PP7, or com sequence, or an L7a ligand, wherein: the ms2 sequence is configured to bind an MCP polypeptide or fragment thereof; the f6 sequence is configured to bind an MCP polypeptide or fragment thereof; the PP7 sequence is configured to bind a PCP polypeptide or fragment thereof; the com sequence is configured to bind a COM polypeptide or fragment thereof; and the L7a ligand is configured to bind an L7a polypeptide or fragment thereof (e.g., RNAB1 and/or RNAB2, see, Russo et al., Biochem J. 2005 Jan. 1; 385(Pt 1):289-99). In some cases, the MCP polypeptide comprises or consists of SEQ ID NO:2, the PCP polypeptide comprises or consists of SEQ ID NO:3, or the COM polypeptide comprises or consists of SEQ ID NO:4. In some cases, the MCP polypeptide comprises or consists of SEQ ID NO:2, the PCP polypeptide comprises or consists of SEQ ID NO:3, and the COM polypeptide comprises or consists of SEQ ID NO:4. In some cases, the L7a polypeptide comprises or consists of SEQ ID NO:16, SEQ ID NO:17, or SEQ ID NO:18 (or an ortholog thereof). In some cases, the ms2 sequence comprises or consists of an RNA encoded by SEQ ID NO:5, the f6 sequence comprises or consists of an RNA encoded by SEQ ID NO:6, the PP7 sequence comprises or consists of an RNA encoded by SEQ ID NO:7, or the com sequence comprises or consists of an RNA encoded by SEQ ID NO:8. In some cases, the L7a ligand comprises or consists of a G rich RNA (e.g., poly-G RNA). In some case, the L7a polypeptide comprises or consists of SEQ ID NO:17 and the L7a ligand comprises or consists of a G rich RNA (e.g., poly-G RNA). In some cases, the ms2 sequence comprises or consists of an RNA encoded by SEQ ID NO:5, the f6 sequence comprises or consists of an RNA encoded by SEQ ID NO:6, the PP7 sequence comprises or consists of an RNA encoded by SEQ ID NO:7, and the com sequence comprises or consists of an RNA encoded by SEQ ID NO:8. In some cases, the 5′ scaffold region and/or the 3′ scaffold region comprises or consists an RNA encoded by of one or more of SEQ ID NO:9, SEQ ID NO:10, SEQ ID NO:11, or SEQ ID NO:12.

In some embodiments, the 5′ scaffold region and/or the 3′ scaffold region is configured to bind one or more, or two or more, polypeptides, and at least one of the polypeptides comprises a restriction endonuclease and an affinity domain having affinity for the 5′ scaffold region or the 3′ scaffold region.

In a second aspect, the present invention provides an expression cassette comprising a promoter (e.g., a heterologous promoter) operably linked to a polynucleotide encoding any one of the foregoing scRNAs. In some embodiments, the heterologous promoter is inducible.

In a third aspect, the present invention provides a method for modulating transcription of a first target nucleic acid comprising: contacting the first target nucleic acid with a first scRNA of any one of the foregoing scRNAs, wherein the first scRNA binds to the first target nucleic acid; or contacting a cell or cell extract containing the first target nucleic acid with a first expression cassette of any one of the foregoing expression cassettes, wherein the first expression cassette contains a polynucleotide encoding the first scRNA, thereby modulating the transcription of the first target nucleic acid.

In some embodiments, the method further comprises contacting the target nucleic acid with a small guide RNA-mediated nuclease (e.g., Cas9, nickase Cas9, or dCas9) or contacting the cell or cell extract with an expression cassette containing a promoter (e.g., a heterologous promoter) operably linked to a polynucleotide encoding a small guide RNA-mediated nuclease (e.g., Cas9, nickase Cas9, or dCas9). In some cases, the method further comprises: contacting a second target nucleic acid with a second structurally different scRNA of any one of the foregoing scRNAs, wherein the second scRNA binds to the second target nucleic acid; or contacting the cell or cell extract, wherein the cell or cell extract contain the first and second target nucleic acid, with a second structurally different expression cassette of any one of the foregoing expression cassettes, wherein the second expression cassette contains a polynucleotide encoding the second scRNA, thereby modulating the transcription of the first and second target nucleic acids. In some cases, the first scRNA activates or represses transcription of the first target nucleic acid and the second scRNA activates or represses transcription of the second target nucleic acid, and the first and second scRNAs exhibit substantially no, or no, cross-talk.

In some cases, the method further comprises: contacting a third target nucleic acid with a third structurally different scRNA of any one of the foregoing scRNAs, wherein the third scRNA binds to the third target nucleic acid; or contacting the cell or cell extract, wherein the cell or cell extract contain the first, second, and third target nucleic acid, with a third structurally different expression cassette of any one of the foregoing expression cassettes, wherein the third expression cassette contains a polynucleotide encoding the third scRNA, thereby modulating the transcription of the first, second and third target nucleic acids. In some cases, the first scRNA activates or represses transcription of the first target nucleic acid, the second scRNA activates or represses transcription of the second target nucleic acid, and the third scRNA activates or represses transcription of the third target nucleic acid, and the first, second, and third scRNAs exhibit substantially no, or no, cross-talk. In some cases, the method further comprises activating or repressing four or more target nucleic acids with four or more structurally different scRNAs, wherein the activation or repression of each target nucleic acid exhibits substantially no, or no, cross-talk with other target nucleic acids.

In a fourth aspect, the present invention provides a kit comprising a first and a second expression cassette, wherein: the first expression cassette comprises a promoter operably linked to a polynucleotide containing a cloning region and a scaffold RNA framework, wherein the scaffold RNA framework comprises: a 5′ scaffold region, wherein the 5′ scaffold region is 5′ of a 3′ scaffold region and specifically binds to at least one 5′ scaffold region binding polypeptide or small molecule; the 3′ scaffold region, wherein the 3′ scaffold region is 3′ of the 5′ scaffold region and specifically binds to at least one 3′ scaffold region binding polypeptide or small molecule; and a transcription termination sequence; and the second expression cassette comprises a promoter operably linked to a small-guide RNA-mediated nuclease.

In some embodiments, the 5′ scaffold region comprises one, two, or more hairpins. In some embodiments, the 3′ scaffold region comprises one, two, or more hairpins. In some embodiments, the 5′ scaffold region and/or the 3′ scaffold region is configured to bind a small guide RNA-mediated nuclease (e.g., Cas9, nickase Cas9, or dCas9). In some cases, the 5′ scaffold region and/or the 3′ scaffold region that is configured to bind a small guide RNA-mediated nuclease comprises a region encoded by SEQ ID NO:1 or SEQ ID NO:13.

In some embodiments, the 5′ scaffold region and/or the 3′ scaffold region is configured to bind two or more polypeptides. In some embodiments, the 5′ scaffold region and/or the 3′ scaffold region is configured to bind one or more, or two or more, polypeptides, and at least one of the polypeptides comprises a transcriptional modulator and an affinity domain having affinity for the 5′ scaffold region or the 3′ scaffold region.

In some embodiments, the 5′ scaffold region and/or the 3′ scaffold region comprises one or more ms2, f6, PP7, com or L7a ligand sequences, wherein: the ms2 sequence is configured to bind an MCP polypeptide or fragment thereof; the f6 sequence is configured to bind an MCP polypeptide or fragment thereof; the PP7 sequence is configured to bind a PCP polypeptide or fragment thereof; the com sequence is configured to bind a COM polypeptide or fragment thereof, and the L7a ligand is configured to bind an L7a sequence or fragment thereof (e.g., RNAB1 or RNAB2).

### BRIEF DESCRIPTION OF THE DRAWINGS

FIG. 1: Genomic Regulatory Programming Using CRISPR and Multi-Domain Scaffolding RNAs. (A) lncRNA molecules are proposed to act as scaffolds to physically assemble epigenetic modifiers at their genomic targets. Modular RNA architectures can encode protein binding domains and DNA targeting sequences to co-localize proteins to genomic loci.

(B) A synthetic CRISPR system using the catalytically inactive dCas9 protein can be repurposed to implement RNA scaffold-based recruitment, allowing simultaneous regulation of independent gene targets. The minimal CRISPRi system silences target genes when dCas9 and an sgRNA assemble to physically block transcription. Fusing dCas9 to transcriptional activators or repressors provides an additional level of functionality. When function is encoded in dCas9 (CRISPRi) or dCas9-fusion proteins, the sgRNA recruits the same function to every target site. To encode both target and function in a scaffold RNA, sgRNA molecules are extended with additional domains to recruit RNA binding proteins that are fused to functional effectors. This approach allows distinct types of regulation to be executed at individual target loci, thus allowing simultaneous activation and repression in the same cell.

FIG. 2: Multiple Orthogonal RNA Binding Modules Can Be Used to Construct CRISPR Scaffolding RNAs. (A) scRNA constructs with MS2, PP7, or com RNA hairpins recruit their cognate RNA-binding proteins fused to VP64 to activate reporter gene expression in yeast. A yeast strain with an unmodified sgRNA and the dCas9-VP64 fusion protein gives comparatively weaker reporter gene activation. The MS2 and PP7 RNA hairpins bind at a dimer interface on their corresponding MCP and PCP binding partner proteins (Chao et al., 2008), potentially recruiting two VP64 effectors to each RNA hairpin. The structure of the com RNA hairpin in complex with its binding protein has not been reported, but functional data suggest that a single Com monomer protein binds at the base of the com RNA hairpin (Wulczyn and Kahmann, 1991). scRNA constructs and corresponding RNA-binding proteins were expressed in yeast with dCas9 and a 1×tetO-VENUS reporter gene.

(B) There is no significant crosstalk between mismatched pairs of scRNA sequences and the incorrect, non-cognate binding proteins. scRNA constructs and RNA-binding proteins were expressed in yeast with dCas9, using a 7×tetO-VENUS reporter gene to detect any potential weak crosstalk between mismatched pairs. Note that the y-axis is on a log-scale and the activity with cognate scRNA-binding protein pairs is significantly greater with the 7×tet reporter compared to the 1× reporter.

(C) Multivalent recruitment with two RNA hairpins connected by a double-stranded linker produces stronger reporter gene activation compared to single RNA hairpin recruitment domains. The 2×MS2 (wt+f6) construct was designed with an aptamer sequence (f6) selected to bind to the MCP protein (Hirao et al., 1998). This construct has two distinct sequences to recruit the same protein, which may help to prevent misfolding between hairpin domains that can occur when two identical hairpins are linked on the same RNA.

(D) A mixed MS2-PP7 scRNA construct constructed using the 2× double-stranded linker architecture recruits both MCP and PCP.

Fold-change values in (A)-(D) are fluorescence levels relative to parent yeast strains lacking scRNA. Values are median±SD for at least three measurements. RNA sequences are reported in Table 1.

FIG. 3: CRISPR RNA Scaffold Recruitment Can Activate or Repress Gene Expression in Human Cells. (A) scRNA constructs with MS2, PP7, or com RNA hairpins recruit corresponding RNA-binding proteins fused to VP64 to activate reporter gene expression in HEK293 cells. scRNA and RNA binding proteins were expressed in a cell line with dCas9 and a TRE3G-EGFP reporter containing a 7× repeat of a tet operator site. For comparison, an unmodified sgRNA targeting the same reporter gene was expressed in a cell line with the dCas9-VP64 fusion protein.

(B) The 2×MS2 (wt+f6) MS2 scRNA construct recruits MCP-VP64 to activate expression of endogenous CXCR4 in HEK293 cells expressing dCas9. Comparatively weak activation is observed in cells with dCas9-VP64 and unmodified sgRNA. There is no significant activation of CXCR4 in cells with dCas9 and unmodified sgRNA. Similar effects were observed at each of three individual target sites located within ˜200 bases of the transcriptional start site (TSS). The three target sites examined are the strongest activation sites from a panel of 10 sites screened in FIG. 8. Cell surface expression of CXCR4 was measured with an APC-coupled anti-human CXCR4 antibody.

(C) The com scRNA construct recruits Com-KRAB to silence a SV40-driven EGFP reporter gene in HEK293 cells expressing dCas9. At the P1 site, upstream of the TSS, recruitment of dCas9 (i.e. CRISPRi) does not silence EGFP, but scRNA-mediated KRAB recruitment does. At the NT1 site, overlapping the TSS, CRISPRi partially silences EGFP, and scRNA-mediated KRAB recruitment enhances silencing relative to CRISPRi. The P1 and NT1 target sites were selected from a panel of sites examined in a prior CRISPR study (Gilbert et al., 2013).

scRNA constructs mediate simultaneous activation and repression at endogenous human genes in HEK293T cells, measured by RT-qPCR. A 2×MS2 (WT+f6) scRNA construct recruits MCP-VP64 to activate CXCR4, and a 1× com scRNA construct recruits COM-KRAB to silence B4GALNT1.

Fold-change values in (A)-(D) are fluorescence levels relative to a parent cell line lacking scRNA. Values are median±SD for at least three measurements. The observed change in CXCR4 mRNA level measured by RTqPCR corresponds to an increased protein level.

FIG. 4: Reprogramming the Output of a Branched Metabolic Pathway with a 3-Gene scRNA CRISPR ON/OFF Switch. (A) Heterologous expression of bacterial violacein biosynthesis pathway in yeast produces violacein from L-Trp following five enzymatic steps and one non-enzymatic step. Branch points at the last two enzymatic transformations catalyzed by VioD and VioC produce four possible pathway outputs.

(B) An scRNA program regulates three genes simultaneously to control flux into the pathway and to direct the choice of product. The yML025 yeast strain (Table 4) has VioBED genes strongly expressed (ON), and VioAC genes weakly expressed (OFF). A 2×PP7 scRNA targets VioA and a 1×MS2 scRNA targets VioC for activation (via recruitment of cognate activator fusion protein). An unmodified sgRNA targets VioD for repression by CRISPRi.

(C) scRNA programs flexibly redirect the output of the violacein pathway. The yML025 yeast strain expressing dCas9, MCP-VP64, and PCP-VP64 was transformed with an empty parent vector (pRS316) or with a plasmid containing one, two, or three scRNA constructs to route the pathway to all four product output states (Table 6). Yeast strains were grown on SD-Ura agar plates. Pathway products were extracted in methanol and analyzed by HPLC. The chromatograms display absorbance at 565 nm.

FIG. 5: The dCas9 Master Regulator Inducibly Executes scRNA-Encoded Programs. (A) dCas9 occupies a central position in scRNA-encoded circuits and can act as a synthetic master regulator. We placed dCas9 under the control of an inducible Gal10 promoter. The yML017 yeast strain (Table 4) has VioABED genes strongly expressed (ON), and VioC weakly expressed (OFF). A 1×MS2 scRNA targets VioC for activation. An unmodified sgRNA targets VioD for repression by CRISPRi.

(B) The presence or absence of the master regulator dCas9 controls execution of the scRNA program. Yeast expressing a two-component scRNA program and MCP-VP64 were grown on agar plates in the presence or absence of galactose to induce dCas9 expression.

When the dCas9 master regulator is not present (−Gal), Vio pathway gene expression remains in the basal state and pathway flux proceeds to the PV product. When dCas9 is present (+Gal), VioC switches ON, VioD switches OFF, and pathway flux diverts to the DV product. The chromatograms display absorbance at 565 nm.

FIG. 6: Encoding Complex dCas9/scRNA Regulatory Programs. scRNAs can be combined with dCas9 to construct designer transcriptional programs in which distinct target genes can be simultaneously activated or repressed, or subject to other types of regulation. Temporal control of the synthetic program can be achieved by inducing the dCas9 protein as a master regulator. Alternative scRNA gene expression programs could be achieved in the same cell by harnessing orthogonal dCas9 proteins that recognize their guide RNAs through distinct sequences (Esvelt et al., 2013). Each orthogonal dCas9 protein could independently control a distinct set of scRNAs, allowing independent control over distinct gene expression programs. The individual scRNAs, in turn, allow independent control at the level of individual genes. The distinct dCas9 proteins could be placed under the control of different extracellular signals or inducible promoters.

FIG. 7. (A) A two base linker between sgRNA and a single MS2 hairpin produces the strongest reporter gene activation. Variable linker-length scRNA constructs were expressed in yeast with dCas9, MCP-VP64, and a 1×tetO-VENUS reporter gene. Expression level is reported as a fold-change in fluorescence relative to a parent yeast strain lacking scRNA. Values are median±SD for at least three measurements.

(B) Increasing numbers of MS2 hairpins give progressively weaker reporter gene activation. One, two, or three MS2 hairpins were connected by two base single-stranded linkers, expressed in yeast and evaluated as described above.

(C) A northern blot for steady-state RNA levels in yeast indicates that RNA levels correlate with functional activity. Increasing linker length or number of MS2 hairpins decreases steady-state RNA levels, with a corresponding decrease in functional activity (FIGS. 7A & B). Steady-state levels for unmodified sgRNA, 1×, and 2×scRNA designs are similar, and the observed activity differences reflect functional differences in the recruitment domains (FIG. 2). The 5′-³²P-labeled DNA oligonucleotide used as a probe hybridizes in the dCas9-binding domain of the sgRNA. Each sgRNA and scRNA construct gives a distinct, three-band pattern that most likely corresponds to read-through of the T₆ terminator sequence (Braglia et al., 2005).

FIG. 8. 10 target sites upstream of the transcriptional start site (TSS) of the human CXCR4 gene were designed (Table 3). Target sites were chosen to hybridize to the non-template (NT) or template (T) strands, immediately downstream of a PAM sequence (NGG), within ˜400 bases of the TSS. Target sites were cloned into a 2× (wt+f6) scRNA construct and evaluated for CXCR4 gene activation in HEK293 cells as described in the main text. For the three sites producing the strongest expression (4, 6, and 10; renamed C1, C2, and C3 respectively), we proceeded to compare scRNA-mediated activation to that with dCas9-VP64 (FIG. 3B). Expression level is reported as a fold-change in fluorescence reporter (an APC-coupled anti-human CXCR4 antibody) relative to a parent cell line lacking scRNA. Values are median±SD for at least three measurements.

FIG. 9: Illustrates the use of an exemplary scRNA binding protein dCas9 as a master regulator in combination with programmable scRNAs and effector proteins fused to scRNA binding molecules to carry out complex RNA-directed gene expression programs. The bottom two panels illustrate the use of such compositions to simultaneously modulate transcription of four different target nucleic acids at differing levels of activation (left) and repression (right) with minimal or no cross-talk.

FIG. 10: Illustrates a schematic diagram of various exemplary scRNA constructs.

### DEFINITIONS

As used in this specification and the appended claims, the singular forms “a,” “an,” and “the” include plural reference unless the context clearly dictates otherwise.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

A “reporter gene” encodes proteins that are readily detectable due to their biochemical characteristics, such as enzymatic activity or chemifluorescent features. One specific example of such a reporter is green fluorescent protein. Fluorescence generated from this protein can be detected with various commercially-available fluorescent detection systems. Other reporters can be detected by staining. The reporter can also be an enzyme that generates a detectable signal when contacted with an appropriate substrate. The reporter can be an enzyme that catalyzes the formation of a detectable product. Suitable enzymes include, but are not limited to, proteases, nucleases, lipases, phosphatases and hydrolases. The reporter can encode an enzyme whose substrates are substantially impermeable to eukaryotic plasma membranes, thus making it possible to tightly control signal formation. Specific examples of suitable reporter genes that encode enzymes include, but are not limited to, CAT (chloramphenicol acetyl transferase; Alton and Vapnek (1979) Nature 282: 864-869); luciferase (lux); β-galactosidase; LacZ; β.-glucuronidase; and alkaline phosphatase (Toh, et al. (1980) Eur. J. Biochem. 182: 231-238; and Hall et al. (1983) J. Mol. Appl. Gen. 2: 101), each of which are incorporated by reference herein in its entirety. Other suitable reporters include those that encode for a particular epitope that can be detected with a labeled antibody that specifically recognizes the epitope.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. In some cases, conservatively modified variants of Cas9 or sgRNA can have an increased stability, assembly, or activity as described herein.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same. For example, a sequence can have at least 80% identity, preferably 85%, 90%, 91%, 92%, 93, 94%, 95%, 96%, 97%, 98%, 99%, or 100% identity, to a reference sequence when compared and aligned for maximum correspondence over a comparison window, or designated region as measured using a sequence comparison algorithm or by manual alignment and visual inspection. Such sequences are then said to be “substantially identical.” With regard to polynucleotide sequences, this definition also refers to the complement of a test sequence. With regard to amino acid sequences, preferably, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence. Yet another indication that two polypeptides are substantially identical is that the two polypeptides retain identical or substantially similar activity.

A “translocation sequence” or “transduction sequence” refers to a peptide or protein (or active fragment or domain thereof) sequence that directs the movement of a protein from one cellular compartment to another, or from the extracellular space through the cell or plasma membrane into the cell. Translocation sequences that direct the movement of a protein from the extracellular space through the cell or plasma membrane into the cell are “cell penetration peptides.” Translocation sequences that localize to the nucleus of a cell are termed “nuclear localization” sequences, signals, domains, peptides, or the like. Examples of translocation sequences include, without limitation, the TAT transduction domain (see, e.g., S. Schwarze et al., Science 285 (Sep. 3, 1999); penetratins or penetratin peptides (D. Derossi et al., Trends in Cell Biol. 8, 84-87); Herpes simplex virus type 1 VP22 (A. Phelan et al., Nature Biotech. 16, 440-443 (1998), and polycationic (e.g., poly-arginine) peptides (Cell Mol. Life Sci. 62 (2005) 1839-1849). Further translocation sequences are known in the art. Translocation peptides can be fused (e.g. at the amino or carboxy terminus), conjugated, or coupled to a compound of the present invention, to, among other things, produce a conjugate compound that may easily pass into target cells, or through the blood brain barrier and into target cells.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize the RNA-mediated nuclease, Cas9 in complex with guide and activating RNA to recognize and cleave foreign nucleic acid.

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. The Cas9 protein can be nuclease defective. For example, the Cas9 protein can be a nicking endonuclease that nicks target DNA, but does not cause double strand breakage. As another example, the Cas9 protein can be unable to nick or cleave target nucleic acid. Such a Cas9 protein is referred to as a dCas9 protein.

As used herein, “activity” in the context of CRISPR/Cas activity, Cas9 activity, scRNA activity, scRNA:nuclease activity and the like refers to the ability to bind to a target genetic element and recruit effector domains to a region at or near the target genetic element. Such activity can be measured in a variety of ways as known in the art. For example, expression, activity, or level of a reporter gene, or expression or activity of a gene encoded by the genetic element can be measured. As another example, a signal (e.g., a fluorescent signal) provided by a recruited effector domain (e.g., a recruited fluorescent protein) can be detected.

As used herein, the term “effector domain” refers to a polypeptide that provides an effector function. Exemplary effector functions include, but are not limited to, enzymatic activity (e.g., nuclease, methylase, demethylase, acetylase, deacetylase, kinase, phosphatase, ubiquitinase, deubiquitinase, luciferase, or peroxidase activity), fluorescence, binding and recruitment of additional polypeptides or organic molecules, or transcriptional modulation (e.g., activation, enhancement, or repression). Thus, exemplary effector domains include, but are not limited to enzymes (e.g., nucleases, methylases, demethylases, acetylases, deacetylases, kinases, phosphatases, ubiquitinases, deubiquitinases, luciferases, or peroxidases), adaptor proteins, fluorescent proteins (e.g., green fluorescent protein), transcriptional enhancers, transcriptional activators, or transcriptional repressors. Adaptor protein effector domains can function to bind, and thus recruit other polypeptides, organic molecules, etc.

### DETAILED DESCRIPTION OF THE INVENTION

### I. Compositions

Described herein are RNAs that contain one or more (e.g., 2, 3, 4, 5, or more) scaffold regions, each scaffold region configured to recruit one or more corresponding scaffold region binding polypeptides or small molecules. Such RNAs that contain one or more scaffold regions are referred to as scaffold RNAs (scRNAs). In some cases, the scaffold region binding polypeptides can be fused to one or more effector domains. In some cases, the scaffold region binding polypeptide is an effector domain as well. For example, the scaffold region binding polypeptide can be an RNA-mediated nuclease, or variant thereof, such as a Cas9 nuclease that binds a scaffold region of the scRNA and possesses nuclease activity. Exemplary scRNA embodiments are schematically illustrated in FIG. 10. The use of a recruitment domain on the 5′ end of the scaffold RNA, as depicted in FIG. 10B, has also been described by Shechner et al., Nat Methods 2015, 12, 664-670.

scRNAs described herein can therefore be useful for recruiting the one or more effector domains to a target nucleic acid, or to a target polypeptide. Multiple scRNAs can be employed, each of which targets a different nucleic acid or polypeptide and/or recruits a different set of effector domains. As described herein, orthogonal scaffold region binding polypeptides, and corresponding effector domains, can be recruited to one or more scRNAs with minimal or no cross-talk between various effector domain functions.

Such scRNAs can be used for a variety of purposes. For example, one or more scRNAs, and corresponding scaffold region binding polypeptides fused to effector domains can be used to construct complex gene expression programs in a variety of different prokaryotic and eukaryotic organisms. As another example, one or more scRNAs, and corresponding scaffold region binding polypeptides fused to effector domains can be used for rapid prototyping of multiple gene perturbations. Such gene perturbations include increasing of expression or decreasing of expression in a constitutive or inducible manner, or a combination thereof. As another example, one or more scRNAs, and corresponding scaffold region binding polypeptides fused to effector domains can be used for metabolic engineering of complex pathways to produce desired products. As yet another example, one or more scRNAs, and corresponding scaffold region binding polypeptides fused to effector domains can be used for cell, or organism, reprogramming or engineering.

scRNAs described herein can be modified by methods known in the art. In some cases, the modifications can include, but are not limited to, the addition of one or more of the following sequence elements: a 5′ cap (e.g., a 7-methylguanylate cap); a 3′ polyadenylated tail; a riboswitch sequence; a stability control sequence; a hairpin; a subcellular localization sequence; a detection sequence or label; or a binding site for one or more proteins. Modifications can also include the introduction of non-natural nucleotides including, but not limited to, one or more of the following: fluorescent nucleotides and methylated nucleotides.

Described herein is a scaffold RNA (scRNA) that contains a nucleic acid binding region. The nucleic acid binding region can be used to localize one or more effector domains to a region at or near the target nucleic acid. In some cases, the nucleic acid binding region is at the 5′ end of the scRNA. Alternatively, the nucleic acid binding region can be at the 3′ end of the scRNA, or in between the 5′ and 3′ ends. In some cases, the scRNA contains a nucleic acid binding region and a scaffold region for recruiting a Cas9 (e.g., dCas9) domain. In such cases, such as when the scRNA is designed to recruit the nuclease activity of a Cas9 domain to a target nucleic acid, the nucleic acid binding region can be 5′ of the Cas9-recruiting scaffold region. Similarly, when the scRNA is designed to recruit a transcriptional repressor activity inherent in dCas9, the nucleic acid binding region can be 5′ of the dCas9 recruiting scaffold region. In other cases, such as when the scRNA is designed to recruit a nuclease deficient dCas9, e.g., a dCas9 domain fused to an effector domain, the nucleic acid binding region can be 5′ of the dCas9 recruiting scaffold region.

The nucleic acid binding region can contain from about 10, 11, 12, 13, 14, or 15 nucleotides to about 16, 17, 18, 19, 20, 21, 22, 23, 24, 25, 26, 27, 28, 29, or 30 nucleotides. In some cases, the binding region of the scRNA is between about 19 and about 21 nucleotides in length. In some cases, the binding region is between about 15 to about 30 nucleotides in length.

Generally, the binding region is designed to complement or substantially complement the target nucleic acid or nucleic acids. In some cases, the binding region can incorporate wobble or degenerate bases to bind multiple nucleic acids. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, G-C content is preferably between about 40% and about 60% (e.g., 40%, 45%, 50%, 55%, 60%). In some cases, if the binding region is at the 5′ end of the scRNA, the binding region can be selected to begin with a sequence that facilitates efficient transcription of the scRNA. For example, the binding region can begin at the 5′ end with a G nucleotide. In some cases, the binding region can contain modified nucleotides such as, without limitation, methylated or phosphorylated nucleotides.

scRNAs described herein contain one or more scaffold regions that each bind, and thereby recruit, one or more scaffold region binding polypeptides. In some cases, the scaffold region binding polypeptides are fused to effector domains. In some cases, the scRNA contains a 5′ scaffold region and a 3′ scaffold region. A 5′ scaffold region refers to a scaffold region that is 5′ of another scaffold region on the same scRNA. A 3′ scaffold region refers to a scaffold region that is 3′ of another scaffold region on the same scRNA. In some cases, the scRNA contains three, four, five, or more scaffold regions. For example, the scRNA can contain, e.g., from 5′ to 3′, a first scaffold region, a second scaffold region, a third scaffold region, a fourth scaffold region, etc. In some cases, scaffold regions of the scRNA are regions containing one or more, or two or more, hairpin, or stem-loop, RNA sequences that can be recognized (e.g., specifically recognized) by one or more corresponding scaffold region binding polypeptides.

In some cases, the scRNA contains a scaffold region that recruits a Cas9 (e.g., dCas9) domain. For example, the scRNA can contain a region encoded by SEQ ID NO:1 or SEQ ID NO:13, and thereby recruit Cas9 (e.g., dCas9) or a Cas9 (e.g., dCas9) fusion protein. In some cases, the scRNA contains a scaffold region that recruits an MCP polypeptide (e.g., SEQ ID NO:2), or a polypeptide containing MCP fused to one or more effector domains. In some cases, the scRNA contains a scaffold region that recruits a PCP polypeptide (e.g., SEQ ID NO:3), or a polypeptide containing PCP fused to one or more effector domains. In some cases, the scRNA contains a scaffold region that recruits a COM polypeptide (e.g., SEQ ID NO:4), or a polypeptide containing COM fused to one or more effector domains. In some cases, the scRNA contains a scaffold region that recruits an L7a polypeptide (e.g., SEQ ID NO:16, 17, or 18, or an ortholog thereof), or a polypeptide containing an L7a polypeptide fused to one or more effector domains.

In some cases, the scaffold region that recruits an MCP polypeptide contains or consists of an ms2 sequence (e.g., encoded by SEQ ID NO:5) or f6 sequence (e.g., encoded by SEQ ID NO:6). In some cases, the scaffold region that recruits an PCP polypeptide contains or consists of a PP7 sequence (e.g., encoded by SEQ ID NO:7). In some cases, the scaffold region that recruits a COM polypeptide contains or consists of a com sequence (e.g., encoded by SEQ ID NO:8). In some cases, the scaffold region that recruits an L7a polypeptide contains or consists of a G-rich RNA region or a poly-G sequence. In some cases, the G-rich RNA region or poly-G sequence contains or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more G nucleotides (e.g., consecutive G nucleotides). In some cases, the G-rich RNA region contains or consists of the foregoing number of G nucleotides and 1, 2, 3, 4, 5, 6, 7, 8, 9, or 10, non-G nucleotides.

In some cases scaffold regions can contain multiple sub-regions to bind multiple scaffold region binding polypeptides. In some cases, such scaffold regions can contain a double-stranded linker between two hairpins, wherein each hairpin binds a scaffold region binding polypeptide. As used herein, such a scaffold region is designated by as “2×ds,” “2×ds,” or the like. For example, ms2-2×ds (or ms2 2×ds or the like) refers to a scaffold region containing two ms2 hairpins separated by a double-stranded linker between the two hairpins. In some cases, the two hairpins separated by a double stranded linker are homologous or identical, as in the example above. In some cases, the two hairpins separated by a double stranded linker are heterologous. In such cases, the two heterologous hairpin sequence names are denoted with the 2×ds. For example, a scaffold region containing f6, a double-stranded linker, and ms2 could be designated ms2-2×ds-f6, or the like.

As such, in some cases, the scaffold region that recruits an MCP polypeptide contains or consists of two ms2 sequences separated by a double-stranded linker (e.g., as encoded by SEQ ID NO:9). In some cases, such an ms2-2×ds sequence can recruit up to four MCP polypeptides because each ms2 sequence can recruit an MCP homodimer. In some cases, the scaffold region that recruits an MCP polypeptide contains or consists of two f6 sequences, such as two f6 sequences separated by a double-stranded linker. In some cases, such an f6 sequence (e.g., f6-2×ds) recruits up to four MCP polypeptides. In some cases, the scaffold region that recruits an MCP polypeptide contains or consists of an ms2 and an f6 sequence separated by a double-stranded linker (e.g., as encoded by SEQ ID NO:10). In some cases, such an ms2-2×ds-f6 sequence recruits up to four MCP polypeptides. In some cases, the scaffold region that recruits an PCP polypeptide contains or consists of two PP7 sequences separated by a double-stranded linker (e.g., as encoded by SEQ ID NO:11). In some cases, such a PP7-2×ds sequence recruits up to four PCP polypeptides. In some cases, the scaffold region contains or consists of an ms2 and a PP7 sequence separated by a double-stranded linker (e.g., as encoded by SEQ ID NO:12). In some cases, such an ms2-2×ds-PP7 sequence recruits one or two MCP polypeptides and one or two PCP polypeptides. Additional combinations of hairpin and double-stranded linkers will be apparent to those of skill in the art. For example, an f6-2×ds-PP7 sequence can be utilized to recruit an MCP (or MCP homodimer) and a PCP (or PCP homodimer) polypeptide to a scaffold region. Similarly, one or more L7a ligands can be utilized in combination with a 2×ds sequence to recruit multiple L7a proteins or fragments thereof, or recruit one or more L7a proteins or fragments thereof and one or more other of the foregoing polypeptides.

scRNAs, as described herein, can be used to recruit a variety of effector domains. Such effector domains can be used to cleave or otherwise modify a target nucleic acid or protein. An exemplary effector domain that can be recruited to a scRNA is Cas9, or a variant or fusion protein thereof. For example, an scRNA containing a Cas9 binding region can be used to recruit Cas9 to a target nucleic acid, thereby cleaving the target nucleic acid in a sequence specific manner. As another example, an scRNA containing a Cas9 binding region can be used to recruit a dCas9 domain fused to another effector domain to a target nucleic acid, thereby modulating the target nucleic acid in a sequence specific manner. The Cas9 (e.g., dCas9) can be fused to one or more copies of a wide variety of effector domains.

The Cas9 protein can be a type I, II, or III Cas9 protein. In some cases, the Cas9 can be a modified Cas9 protein. Cas9 proteins can be modified by any method known in the art. For example, the Cas9 protein can be codon optimized for expression in host cell or an in vitro expression system. Additionally, or alternatively, the Cas9 protein can be engineered for stability, enhanced target binding, or reduced aggregation.

The Cas9 can be a nuclease defective Cas9 (i.e., dCas9). For example, certain Cas9 mutations can provide a nuclease that does not cleave or nick, or does not substantially cleave or nick the target sequence. Exemplary mutations that reduce or eliminate nuclease activity include one or more mutations in the following locations: D10, G12, G17, E762, H840, N854, N863, H982, H983, A984, D986, or A987, or a mutation in a corresponding location in a Cas9 homologue or ortholog. The mutation(s) can include substitution with any natural (e.g., alanine) or non-natural amino acid, or deletion. An exemplary nuclease defective dCas9 protein is Cas9D10A&H840A (Jinek, et al., Science. 2012 Aug. 17; 337(6096):816-21; Qi, et al., Cell. 2013 Feb. 28; 152(5):1173-83).

dCas9 proteins that do not cleave or nick the target sequence can be utilized in combination with an scRNA, such as one or more of the scRNAs described herein, to form a complex that is useful for targeting, detection, or transcriptional modulation of target nucleic acids as further explained below. The dCas9 can be targeted to one or more genetic elements by virtue of the nucleic acid binding regions encoded on one or more scRNAs. Recruitment of dCas9 can therefore provide recruitment of additional effector domains as provided by polypeptides fused to the dCas9 domain. For example, a polypeptide comprising an effector domain can be fused to the N and/or C-terminus of a dCas9 domain. In some cases, the polypeptide encodes a transcriptional activator or repressor. In some cases, the affinity agent is fused to one or more copies of an effector domain, such as an enzyme (e.g., a nuclease, a methylase, a demethylase, an acetylase, a deacetylase, a kinase, a phosphatase, a ubiquitinase, a deubiquitinase, a luciferase, or a peroxidase), a fluorescent protein (e.g., a green fluorescent protein), a transcriptional enhancer, a transcriptional activator, or a transcriptional repressor.

In one embodiment, the dCas9 is a transcriptional activator and comprises a dCas9 domain and transcriptional activator domain. In some cases, the dCas9 domain is fused to two or more copies of a p65 activation domain (p65AD). In some cases, the dCas9 domain transcriptional activator comprises a dCas9 domain fused to two or more, three or more, or four or more copies of a VP16 or VP64 activation domain. In some cases, the dCas9 domain is fused to at least one copy of a first activation domain (e.g., p65AD) and at least one copy of a second activation domain (e.g., VP16 or VP64).

In some embodiments, the dCas9 is a transcriptional repressor and comprises a dCas9 domain and a transcriptional repressor domain. In some cases, the dCas9 domain is fused to one or more or two or more copies of a Krüppel associated box (KRAB) repressor domain. In some cases, the dCas9 domain is fused to one or more or two or more copies of a chromoshadow domain (CSD) repressor. In some cases, the dCas9 is fused to at least one copy of a first repressor domain (e.g., a KRAB domain) and at least one copy of a second repressor domain (e.g., a CSD domain).

In some embodiments, effector domains, such as any of the effector domains described herein, can be fused to a scaffold region binding polypeptide. Such scaffold region binding polypeptide-effector domain fusions can be recruited to an scRNA, and thereby recruited to a target nucleic acid or target polypeptide. For example, an MCP polypeptide can be fused to any one or more of the effector domains described herein. As another example, a PCP polypeptide or a COM polypeptide can be fused to any one or more of the effector domains described herein. As another example, an L7a protein (e.g., SEQ ID NO:16 or an ortholog thereof) or fragment thereof (e.g., SEQ ID NO:17 or 18) can be fused to any one or more of the effector domains herein.

In some cases, the effector domain fused to Cas9 (e.g., dCas9), or any other scaffold region binding polypeptide, is an enzyme (e.g., a nuclease, a methylate, a demethylase, an acetylase, a deacetylase, a kinase, a phosphatase, a ubiquitinase, a deubiquitinase, a luciferase, or a peroxidase), a fluorescent protein (e.g., a green fluorescent protein), a chromatin modifier, a transcriptional enhancer, a transcriptional activator, or a transcriptional repressor. Exemplary chromatin modifiers include enzymes that methylate or demethylate DNA or histones, or enzymes that acetylate or deacetylate histones. Exemplary transcriptional repressors include Krüppel associated box (KRAB) repressor domains and chromoshadow domain (CSD) repressors. Exemplary transcriptional activators include Herpes Simplex Virus Viral Protein 16 (VP16) domains. Exemplary transcriptional activators also can include tandem arrays of VP16 domains. For example, the VP64 domain, which consists of four tandem arrays of VP16 can be used as a transcriptional activator effector domain.

In some embodiments, the scaffold regions bind one or more scaffold region binding polypeptides and one or more small molecules. In some cases, the small molecules can bind to one or more scaffold regions and competitively, non-competitively, or allosterically modulate (e.g., inhibit or permit) binding of the scaffold region binding polypeptide to the scaffold region. In some cases, the small molecules can bind to one or more scaffold regions and induce or stabilize a scaffold region conformation that favors or allows binding of a scaffold region binding polypeptide. Thus, an organism, cell, or cell extract can be treated with a small molecule to modulate the activity of the scRNA by modulating recruitment of scaffold region binding polypeptides, and thereby modulating recruitment of effector domains fused to such polypeptides, to target nucleic acids or polypeptides.

In some cases, the small molecules have a molecular weight of less than about 5,000; less than about 1,000; or less than about 500 daltons. In some cases, the small molecules have a c Log P or a log P of 5 or less. In some cases, the small molecules have a log P or c Log P of from −0.4 to 5.6. In some cases, the small molecules have no more than 5, or 10, hydrogen bond donors or acceptors. In some cases the small molecules have 10 or fewer rotatable bonds. In some cases, the small molecules have a polar surface equal to or less than 140 Å². In some cases, the small molecules have a molar refractivity of from 40 to 130. Exemplary small molecules that can bind a scaffold region include, but are not limited to tetracycline or theophylline.

scRNAs described herein can contain a region that encodes a transcriptional termination region. The transcriptional termination region can contain or consist of a wide variety of transcriptional termination sequences. An exemplary transcriptional termination sequence is seven consecutive uracil nucleotides (e.g., encoded by SEQ ID NO:14) or a SUP4 terminator (e.g., encoded by SEQ ID NO:15).

Also described herein are expression cassettes or vectors for producing one or more RNAs or polypeptides described herein. Such expression cassettes or vectors can be used for producing one or more scRNAs described herein in a host organism, cell, or cell extract. The expression cassettes can contain a promoter (e.g., a heterologous promoter) operably linked to a polynucleotide encoding an scRNA. In some cases, the polynucleotide encoding the scRNA of the expression cassette further encodes one or more scaffold region binding polypeptides. In some cases, one or more expression cassettes that do not encode an scRNA can be used to generate one or more scaffold region binding polypeptides. Such an expression cassette can contain a promoter (e.g., a heterologous promoter) operably linked to a polynucleotide encoding one or more scaffold region binding polypeptides.

The promoter selected for any of the expression cassettes described herein can be inducible or constitutive. The promoter can be tissue specific. In some cases, the promoter is a strong promoter. For example, the promoter can be a CMV promoter, an SFFV long terminal repeat promoter, or the human elongation factor 1 promoter (EF1A). In some cases, the promoter is a weak promoter as compared to the human elongation factor 1 promoter (EF1A). In some cases, the promoter is a weak mammalian promoter. In some cases, the weak mammalian promoter is a ubiquitin C promoter, a vav promoter, or a phosphoglycerate kinase 1 promoter (PGK). In some cases, the weak mammalian promoter is a TetOn promoter in the absence of an inducer. In some cases, when a TetOn promoter is utilized, the host organism, cell, or cell extract is also contacted with a tetracycline transactivator. In some cases, the promoter is an SNR52 promoter or a U6 promoter. For example, a U6 or H1 PolIII promoter operable in mammalian (e.g., human) cells can be selected to, e.g., drive expression of an scRNA or other construct. For example, the SNR52 PolIII promoter operable in fungal (e.g., yeast) cells can be selected to, e.g., drive expression of an scRNA. In some cases, a PolIII promoter is advantageous for scRNA expression due to the precise initiation and termination of transcription provided by PolIII.

In some embodiments, the strength of the selected scRNA promoter can selected to express an amount of scRNA that is proportional to the amount of scaffold region binding polypeptide or scaffold region binding polypeptide expression. In some embodiments, the strength of the selected promoter is selected to modulate, or titrate, the activity of the scRNA against a target nucleic acid or target polypeptide. For example, if the scRNA targets a gene and recruits a transcriptional repressor or activator, the strength, or level of induction, of the scRNA promoter can be selected to achieve a desired level of transcriptional repression or activation.

Similarly, the strength of a selected promoter operably linked to a scaffold region binding polypeptide can be selected to be proportional to the amount of corresponding scaffold regions or proportional to the expression level of corresponding scaffold regions. In some cases, the expression level of the scaffold region binding polypeptides is modulated to modulate, or titrate, the activity of one or more effector domains fused to the scaffold region binding polypeptide. For example, if an scRNA targets a gene and recruits a scaffold region binding polypeptide fused to a transcriptional repressor or activator, the strength, or level of induction, of a scaffold region binding polypeptide promoter can be selected to achieve a desired level of transcriptional repression or activation.

In some cases, an expression cassette is provided for cloning a nucleic acid binding region of interest in frame with one or more scaffold regions (e.g., 3′ and/or 5′ scaffold regions). In some cases, the expression cassette for cloning a nucleic acid binding region of interest in frame with one or more scaffold region comprises a polynucleotide encoding a Cas9 (e.g., dCas9) recruiting scaffold region. In some cases, cloning region for insertion of a nucleic acid binding region is 5′ of the polynucleotide encoding a Cas9 recruiting scaffold region.

The expression cassette can include one or more localization sequences. The expression cassette can be in a vector, such as a plasmid, a viral vector, a lentiviral vector, etc. In some cases, the expression cassette is in a host cell. The expression cassette can be episomal or integrated in the host cell.

### II. Methods

Described herein are methods for recruiting one or more effector domains to a target nucleotide or a target nucleic acid with an scRNA. For example, an scRNA containing a nucleic acid binding region and one or more scaffold regions can be used to recruit corresponding scaffold region binding polypeptides and their effector domains to the target nucleic acid. Such an scRNA can, e.g., be utilized to recruit transcriptional activators or repressors to modulate transcription of the target nucleic acid.

The recruiting can be performed in vivo, e.g., in a cell, or in vitro, e.g., in a cell extract. In one embodiment, the recruiting is performed in a cultured cell. In some embodiments, the recruiting is performed by contacting a cell (e.g., a cell in culture or a cell in an organism) or cell extract with a composition containing an scRNA and one or more scaffold region binding polypeptides (e.g., dCas9, MCP, PCP, COM, L7a, or a fragment or ortholog thereof). In some cases, at least one of the scaffold region binding polypeptide is a Cas9 (e.g., dCas9) protein. In some cases, the one or more scaffold region binding peptides are fused one or more effector domains or one or more copies of an effector domain. The method can include recruiting 2, 3, 4, 5, 6, 7, 8, 9, 10, 11, 12, 13, 14, 15, 16, 17, 18, 19, 20, 21, 22, 23, 24, or more scaffold region binding polypeptides, and their fused effector domains to the target nucleic acid or target polypeptide.

The contacting can be performed by contacting the cell or cell extract with one or more expression cassettes that contain a promoter operably linked to a polynucleotide that encodes one or more components of the composition. In some cases, each component of the composition is encoded in a polynucleotide in a separate expression cassette. In some cases, an expression cassette can contain one or more polynucleotides that encode multiple components of the composition. In some cases, one or more of the expression cassettes are in a vector, such as a lentiviral vector. For example, a cell or population of cells can be transiently or stably transfected with a vector (e.g., lentiviral vector) containing an expression cassette having a promoter operably linked to a polynucleotide encoding an scRNA. As another example, a cell or population of cells can be transiently or stably transfected with a vector (e.g., lentiviral vector) containing an expression cassette having a promoter operably linked to a polynucleotide encoding one or more scaffold region binding polypeptides (e.g., dCas9, MCP, PCP, COM, L7a, or a fragment or ortholog thereof, or any other scaffold region binding polypeptide). In some cases, the scaffold region binding polypeptide is fused to one or more effector domains.

The cell or population of cells can be contacted or transfected with a first expression cassette, and optionally subjected to a selection step to select against a cell that has not been transfected. Stably or transiently transfected cells can be transfected with a second vector (e.g., lentiviral vector) containing an expression cassette with a promoter operably linked to a polynucleotide encoding a different scRNA, or a different scaffold region binding polypeptide, or the like. Additional steps can be performed to contact the cell with additional scRNAs or scaffold region binding polypeptides. One of skill in the art can appreciate that expression vectors described herein can be used in any order, or simultaneously to contact a cell or cell extract with an scRNA or a scaffold region binding polypeptide. For example a cell can be first transfected with an expression vector with a promoter operably linked to a polynucleotide encoding an scRNA and then transfected with an expression vector with a promoter operably linked to a polynucleotide encoding a dCas9 fused to one or more effector domains.

In some cases, multiple scaffold RNAs, each binding multiple orthogonal scaffold region binding polypeptides can be used simultaneously in the same cell to modulate transcription of multiple target nucleic elements with little or no cross-talk. As such, the methods can be used to carry out complex gene expression programs in which multiple genes are turned off and on independently. In some cases, inducible promoters can be utilized for one or more scRNAs, or one or more scaffold region binding polypeptides to provide temporal control.

### III. Kits

Also described herein are kits for performing methods described herein or obtaining or using a composition described herein. Such kits can include one or more polynucleotides encoding one or more compositions described herein (e.g., an scRNA, a dCas9, a scaffold region binding polypeptide such as MCP, PCP, COM, L7a, or a fragment or ortholog thereof), or one or more effector domains, or portions thereof. The polynucleotides can be provided as expression cassettes with promoters operably linked to one or more of the foregoing polynucleotides. The expression cassettes can be provided in one or more vectors for transfecting a host cell. In some embodiments, the kits provide a host cell transfected with one or more polynucleotides encoding one or more compositions described herein.

For example, a kit can contain a vector containing an expression cassette with a promoter operably linked to a polynucleotide encoding an scRNA backbone and a cloning region. A nucleic acid binding region of the scRNA can be cloned into the cloning region, thereby generating a polynucleotide encoding an scRNA that targets a desired genetic element. Alternatively, or in addition, the kit can contain an expression cassette with a promoter operably linked to a polynucleotide encoding an scRNA. As another example, a kit can contain a vector containing an expression cassette with a promoter operably linked to a polynucleotide encoding a cloning region and one or more effector domains. A polynucleotide encoding a scaffold region binding polypeptide (e.g., Cas9, dCas9, COM, MCP, PCP, L7a, or a fragment or ortholog thereof) can be cloned into the cloning region thereby fusing the scaffold region binding polypeptide to the one or more effector domains.

In one embodiment, the kit contains (i) an expression cassette with a heterologous promoter operably linked to a polynucleotide encoding an affinity agent fusion protein, wherein the affinity agent fusion protein comprises: an affinity domain that specifically binds the epitope; and a effector domain; and/or (ii) an expression cassette encoding: (a) a heterologous promoter, a cloning site, and a multimerized epitope, wherein the cloning site is configured to allow cloning of a polypeptide of interest operably linked to the promoter and fused to the multimerized epitope; or (b) a heterologous promoter operably linked to a polypeptide of interest fused to a multimerized epitope.

All patents, patent applications, and other publications, including GenBank Accession Numbers, cited in this application are incorporated by reference in the entirety for all purposes.

### EXAMPLES

The following examples are provided by way of illustration only and not by way of limitation. Those of skill in the art will readily recognize a variety of non-critical parameters that could be changed or modified to yield essentially the same or similar results.

### Example 1

### Introduction

Eukaryotic cells achieve many different states by executing complex transcriptional programs that allow a single genome to be interpreted in numerous, distinct ways. In such expression programs, specific loci throughout the genome must be regulated independently. For example, during development, it is often critical to not only activate sets of genes associated with a new cell fate, but also to simultaneously repress or silence sets of genes associated with maintaining a prior or alternative fate. Similarly, environmental conditions often trigger shifts in a cell's metabolic state, which requires activating expression of a new set of enzymes and repression of other previously expressed enzymes, leading to new metabolic fluxes. This kind of complex multi-locus, multi-directional expression program is encoded largely by the pattern of transcriptional activators, repressors, or other regulators that assemble at distinct sites in the genome. Reprogramming these instructions to produce a different cell type or state thus requires precisely targeted changes in gene expression over a broad set of genes.

How might we engineer novel gene expression programs that match the sophistication of natural programs? Such capabilities would provide powerful tools to probe how changes in gene expression programs lead to diverse cell types. These tools would also provide the ability to engineer more sophisticated designer cell types for therapeutic or biotechnological applications. Although a number of new transcriptional engineering platforms have recently been developed, these present major constraints in achieving the goal of constructing complex transcriptional programs. For example synthetic transcription factors (such as designed zinc fingers or TAL effectors) can be used to target a specific regulatory action to a key genomic locus, but it is challenging to simultaneously target many loci in parallel, because each DNA-binding protein must be individually designed and tested (Gaj et al., 2013). The bacterial type II CRISPR (clustered regularly interspaced short palindromic repeats) interference system (CRISPRi) provides an alternative suite of tools for genome regulation (Qi et al., 2013). In particular, a catalytically inactive Cas9 (dCas9) protein which lacks endonuclease activity can be used as a DNA recognition platform that can flexibly target many loci in parallel, by using Cas9 binding guide RNAs that recognize target sequences based only on predictable Watson-Crick base pairing. This CRISPRi regulation can be used to achieve activation or repression by fusing dCas9 to activator or repressor modules (Gilbert et al., 2013; Mali et al., 2013a), but these direct protein fusions are constrained to only one direction of regulation. Thus it remains challenging to engineer regulatory programs in which many loci are targeted simultaneously, but with distinct types of regulation at each locus.

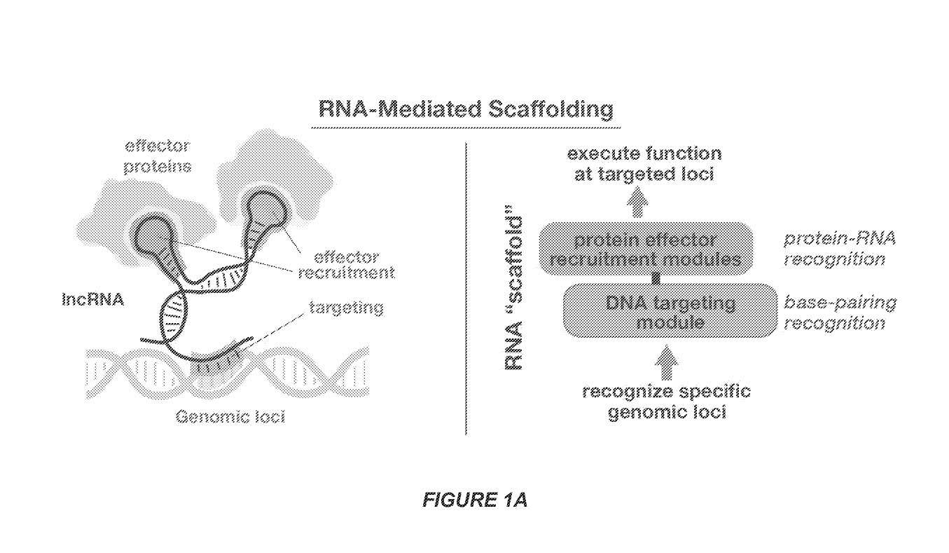

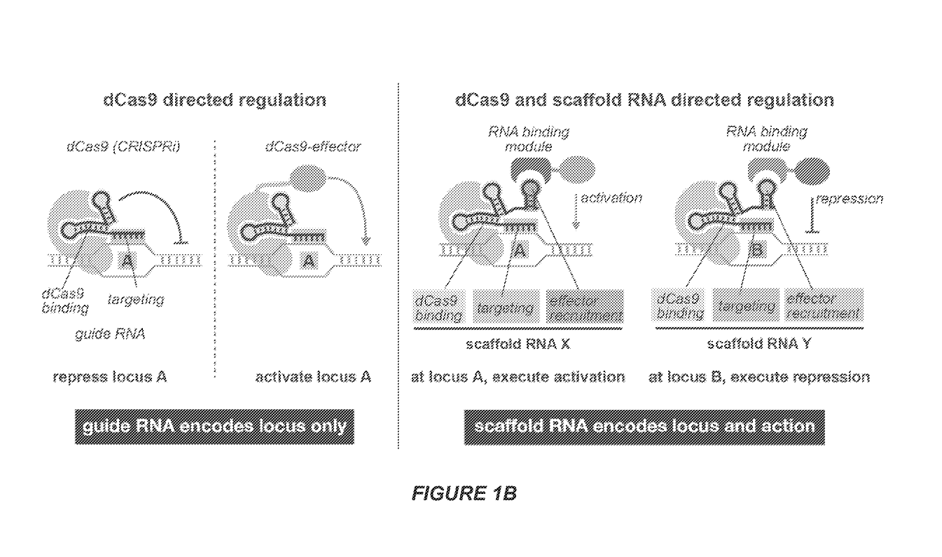

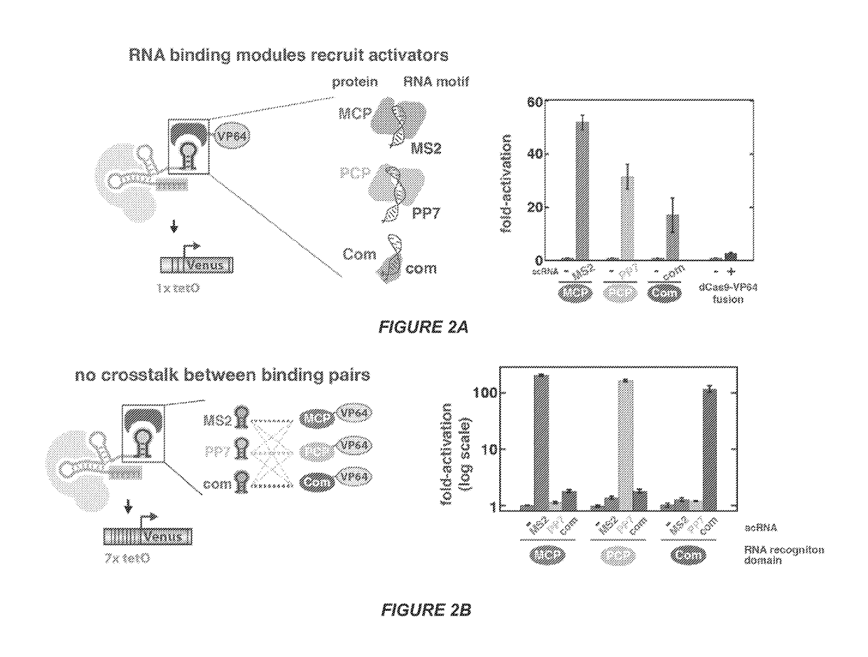

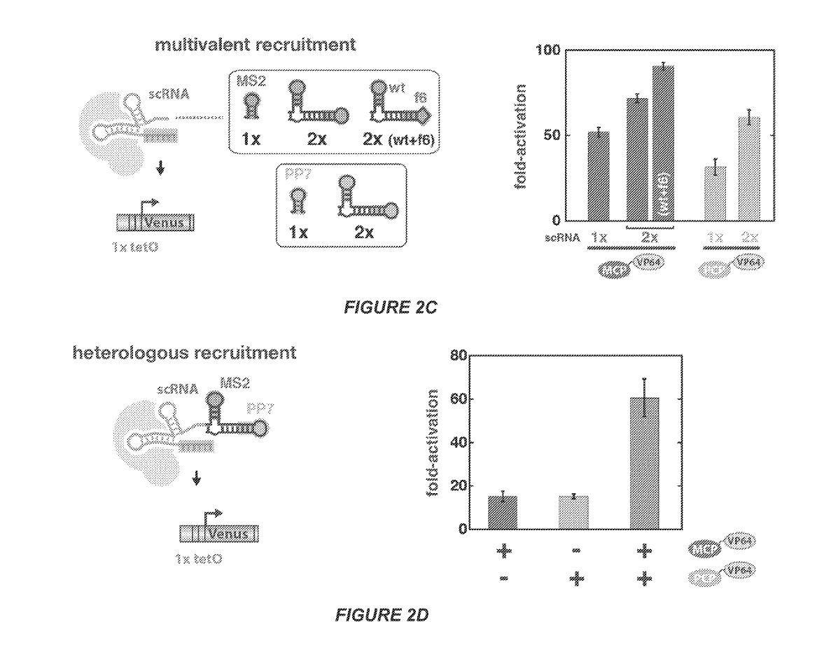

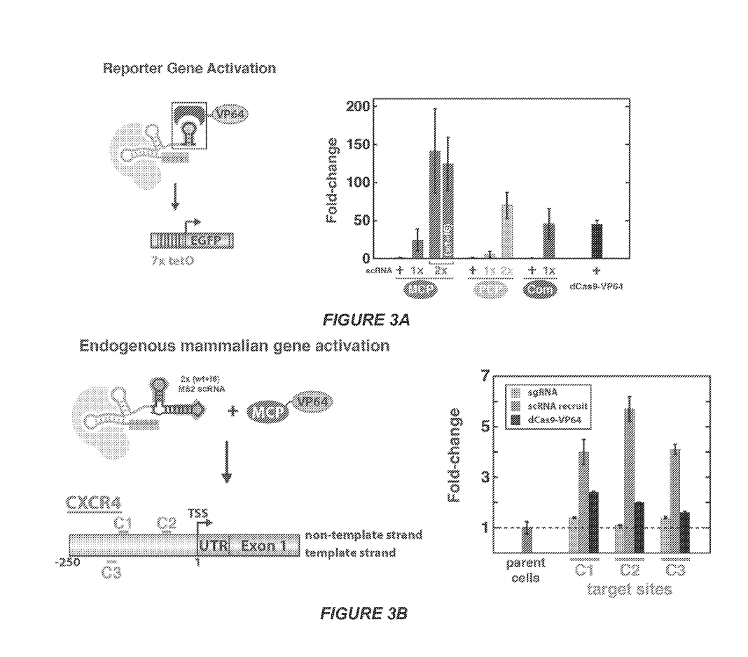

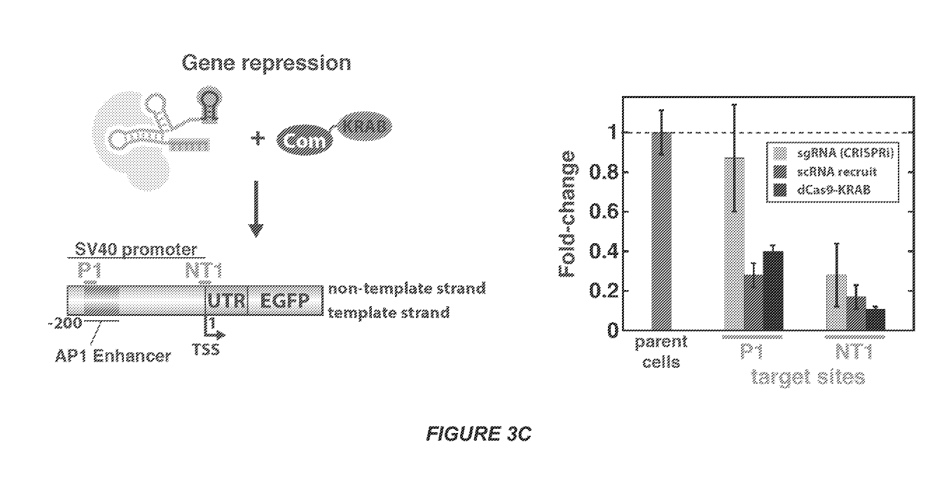

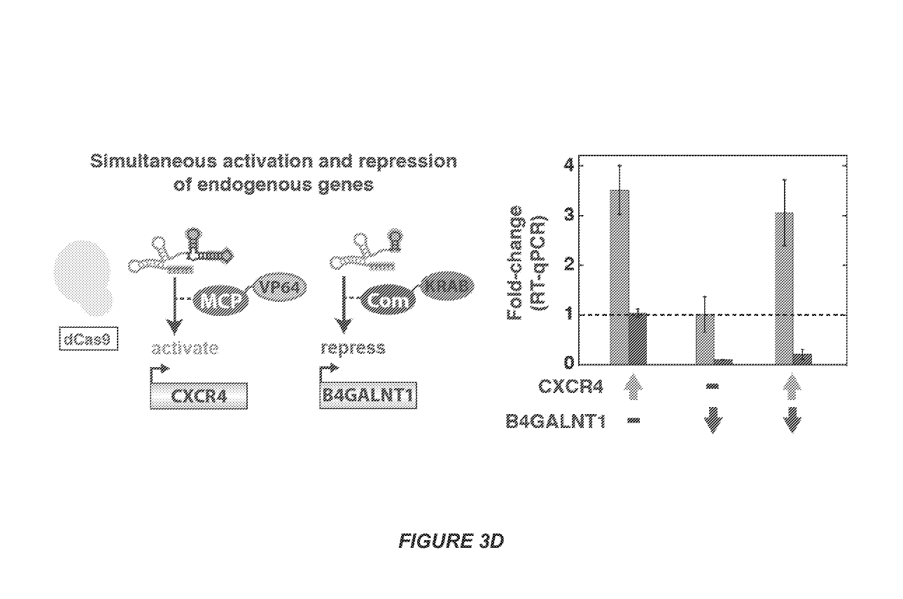

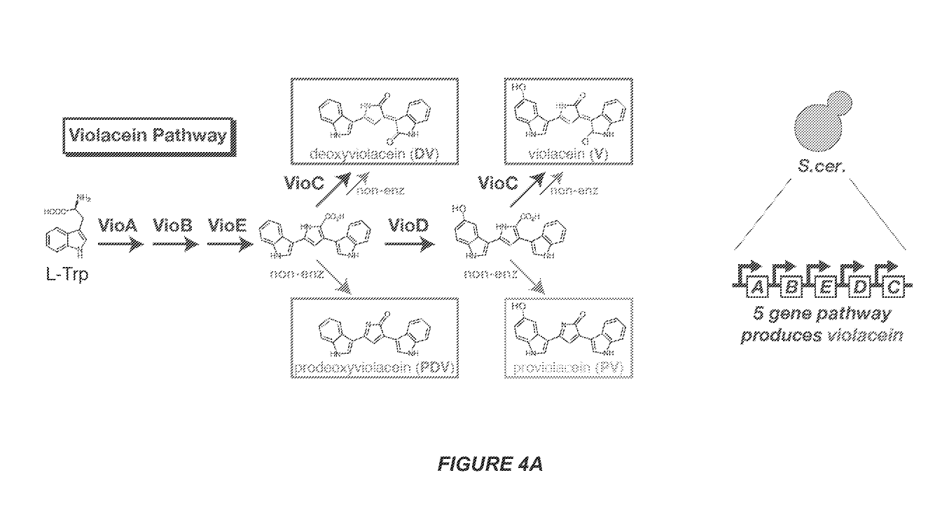

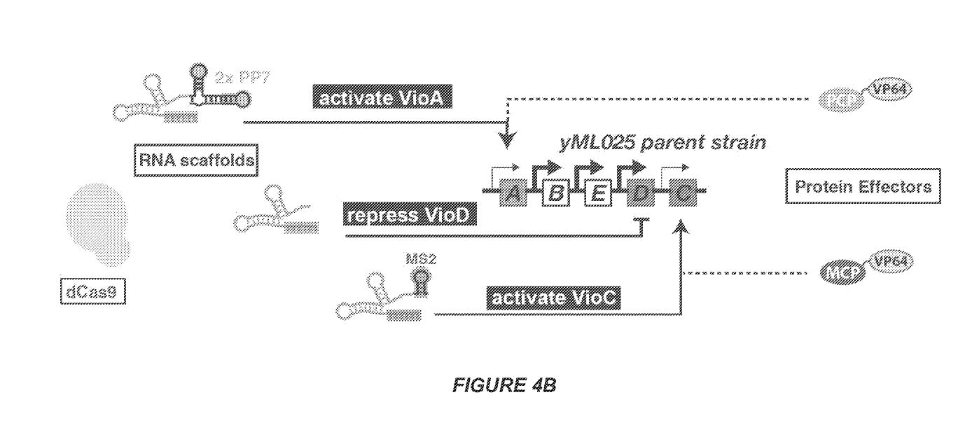

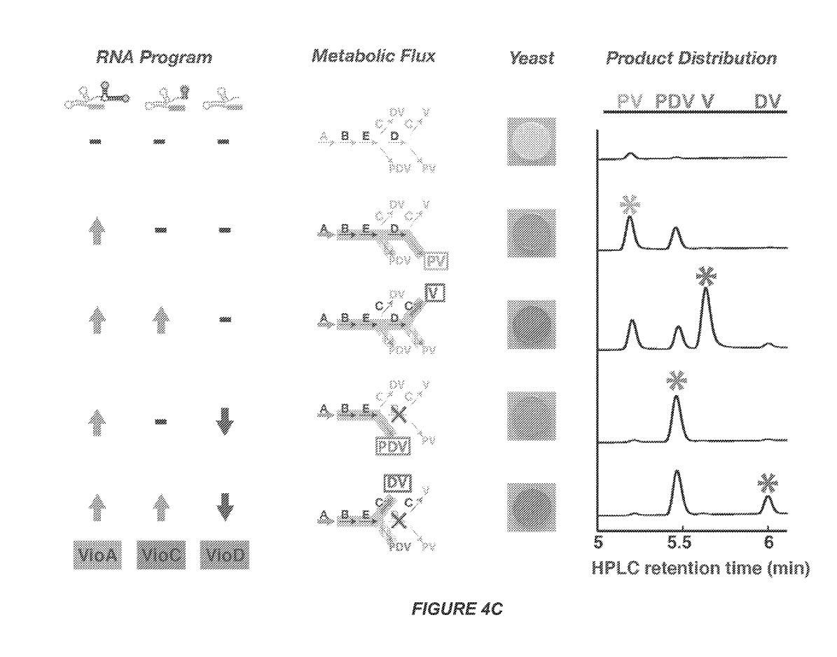

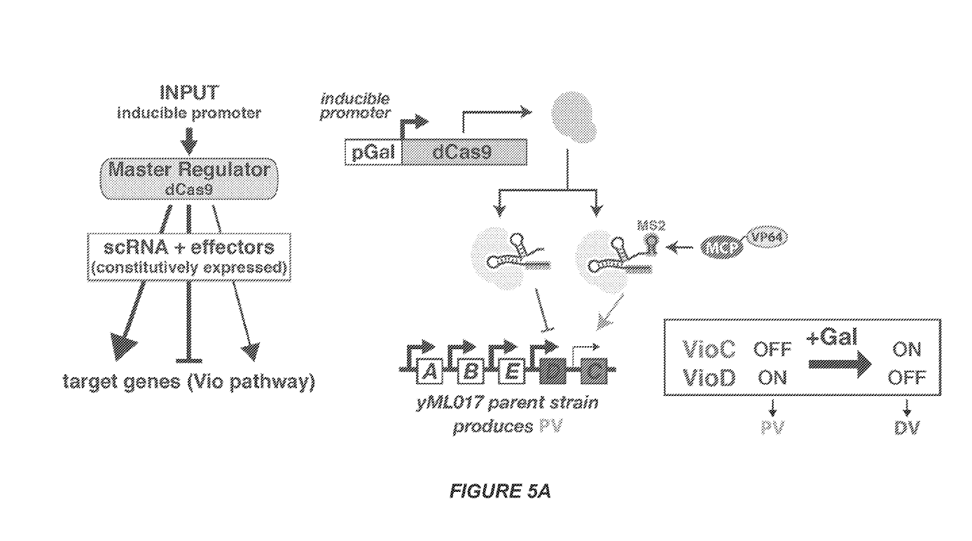

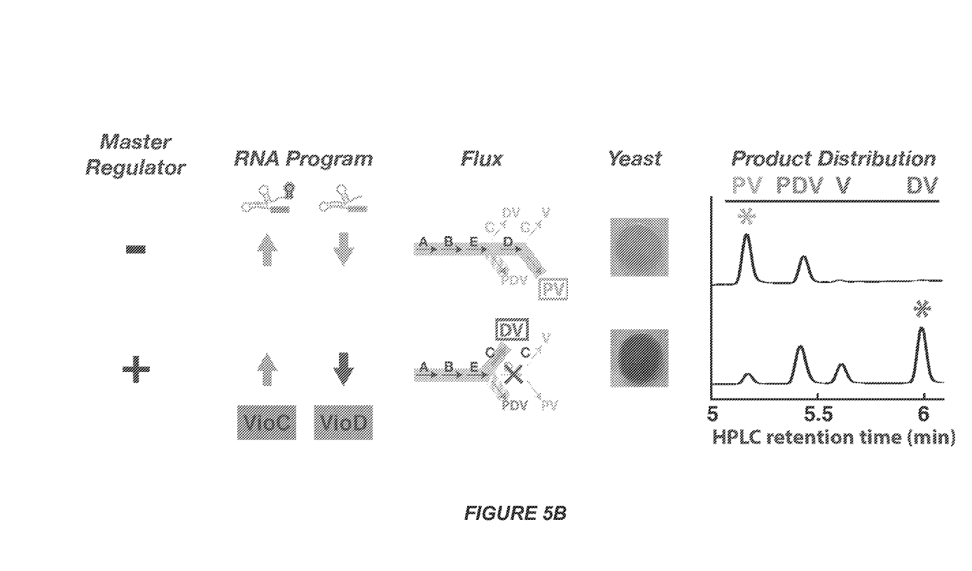

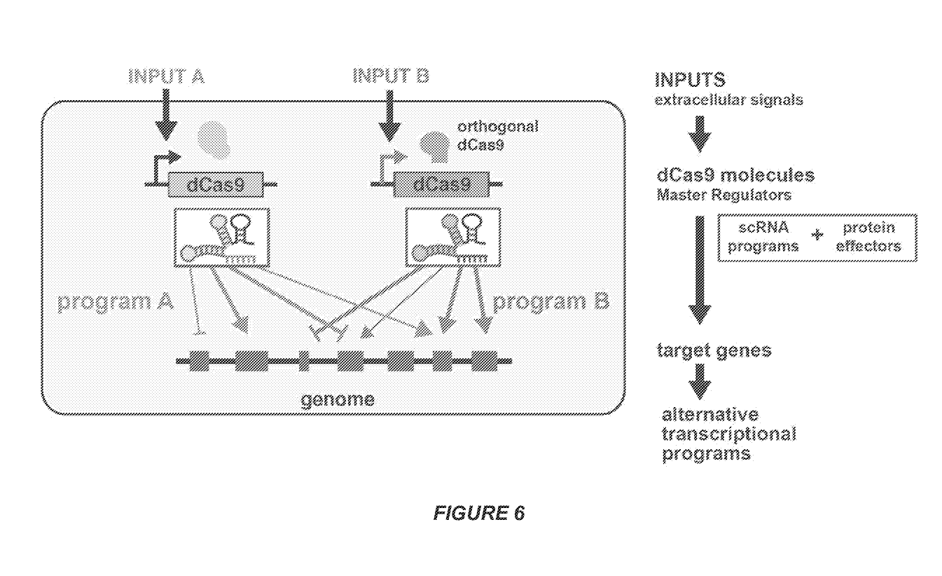

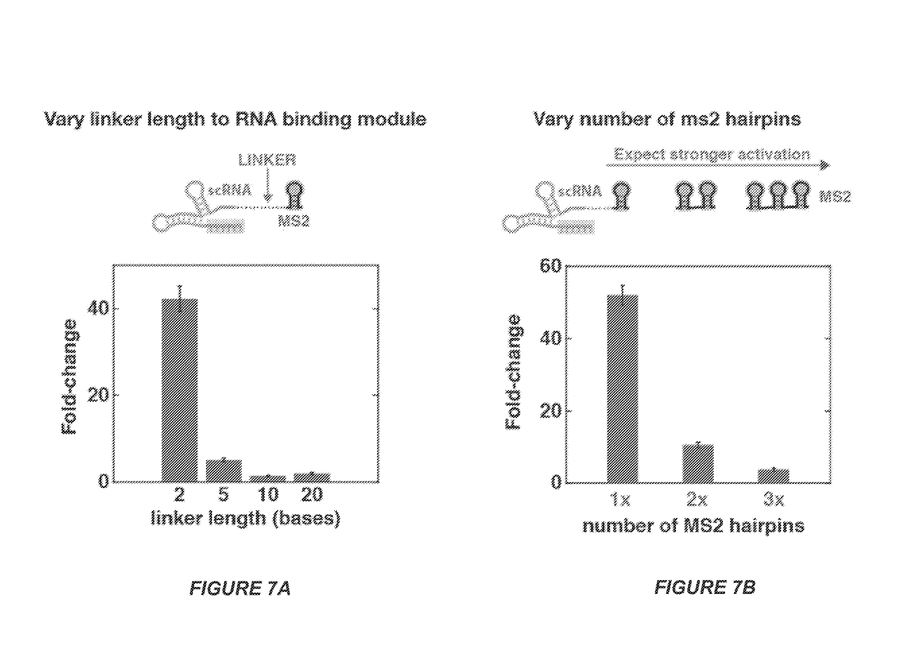

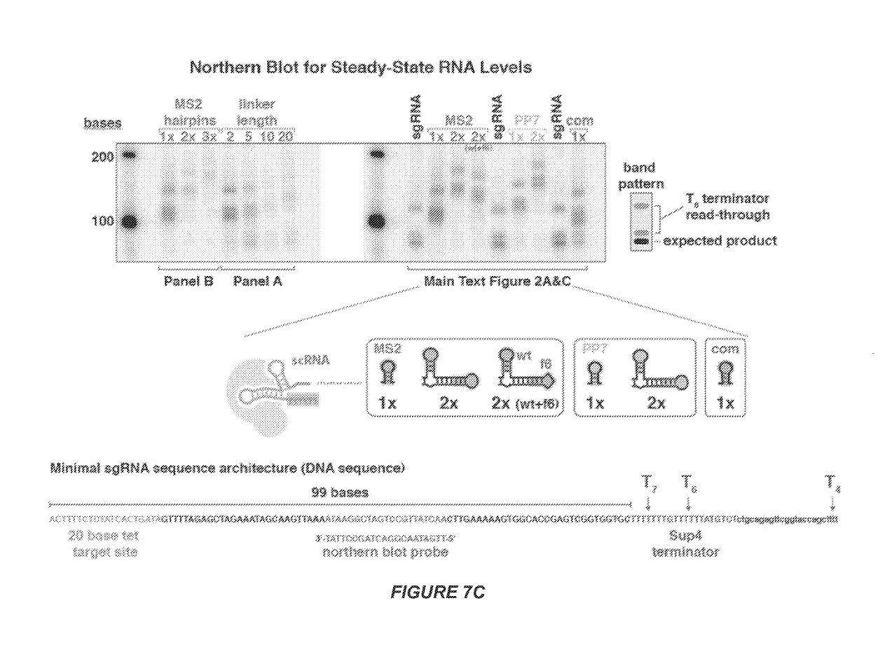

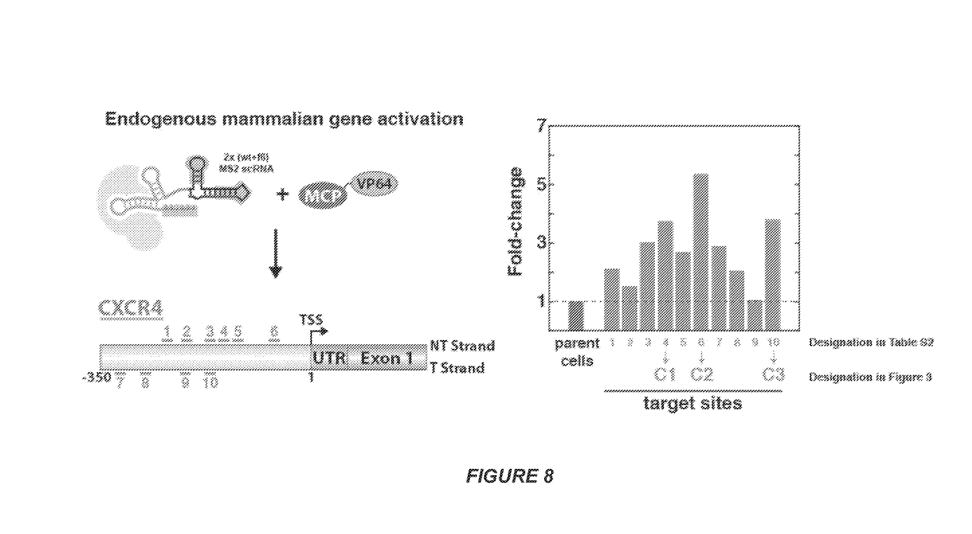

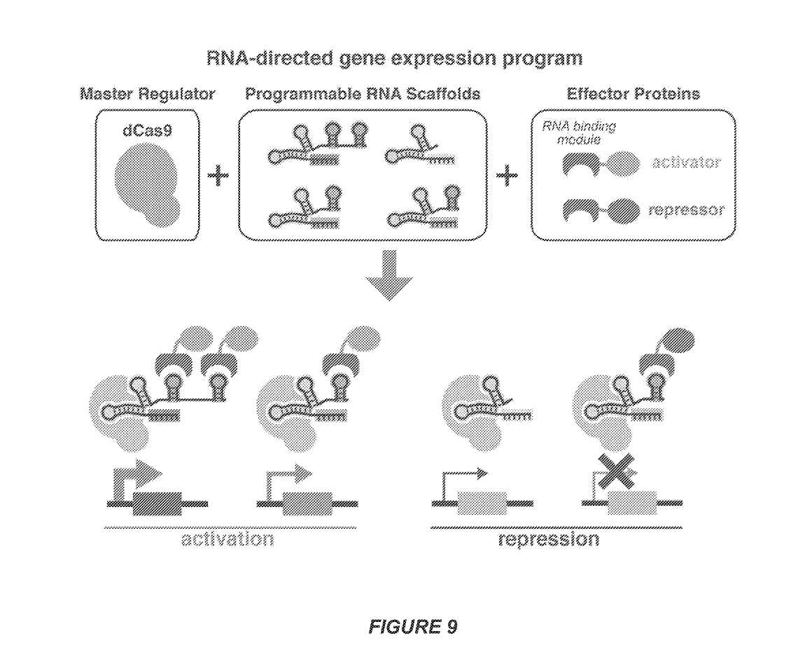

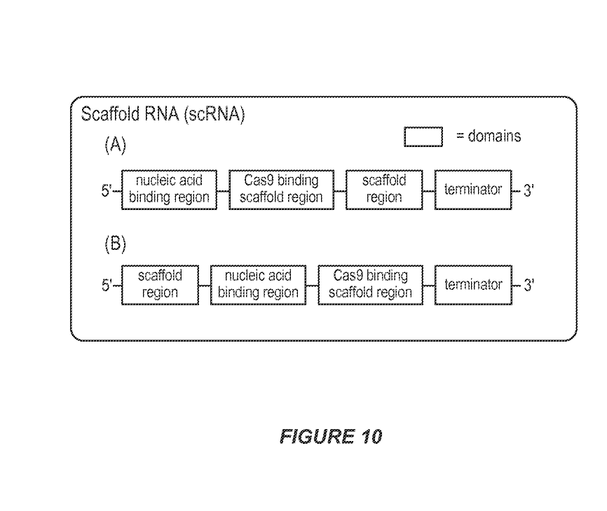

To develop a more flexible platform for synthetic genome regulation that allows locus-specific action, we took inspiration from natural regulatory systems that have a more modular organization to encode both target and function in the same molecule. In cell signaling pathways, scaffold proteins act to physically assemble functionally interacting components so that key functional outcomes can be precisely controlled in time and space (Good et al., 2011). Similar fundamental scaffolding principles apply in genome organization, where, for example, long non-coding RNA (lncRNA) molecules are proposed to act as assembly scaffolds that recruit key epigenetic modifiers to specific genomic loci (FIG. 1A) (Rinn and Chang, 2012; Spitale et al., 2011). The idea that RNA can be used to coordinate biological assemblies has important implications for engineering. RNA is inherently modular and programmable: DNA targets can be recognized by base pairing, and modular RNA-protein interaction domains can be used to recruit specific proteins (FIG. 1A). The ability of engineered RNA scaffolds to coordinate functional protein assemblies has already been elegantly demonstrated (Delebecque et al., 2011).

To implement a synthetic, modular RNA-based system for locus-specific transcriptional programming, we can extend the CRISPR small guide RNA (sgRNA) sequence with modular RNA domains that recruit RNA-binding proteins. This approach converts the sgRNA into a scaffold RNA (scRNA) that physically links DNA binding and protein recruitment activities into one molecule (FIG. 1B). Critically, a single scRNA molecule can thus encode both information about the target locus and instructions about what regulatory function should be executed at that locus. Thus, because both target and function are encoded in the RNA, this approach allows multidirectional regulation (i.e., simultaneous activation and repression) of different target genes as part of the same regulatory program in the same cell. Engineering multivalent RNA recruitment sites on each scRNA offers the further possibility of independently tuning the strength of activation or repression at each individual target site. The potential viability of this approach is supported by a recent report showing that a sgRNA extended with MS2 hairpins can recruit activators to a reporter gene in human cells (Mali et al., 2013a).

Here, we demonstrate that CRISPR sgRNAs can be repurposed as scaffolding molecules to recruit transcriptional activators or repressors, thus enabling rapid and parallel programmable locus-specific regulation. We use the budding yeast S. cerevisiae as a testbed to identify 3 orthogonal RNA-protein binding modules and to optimize scRNA designs for single and multivalent recruitment sites. We show that the system developed in yeast also functions efficiently in human cells to regulate reporter and endogenous target sites, and we extend its scope to include recruitment of chromatin modifiers for gene repression. We then demonstrate that we can use a set of CRISPR scaffold RNA molecules as the instructions to construct multiple synthetic gene expression programs. Specifically we are able to regulate multiple genes in a highly-branched biosynthetic pathway in yeast such that key enzymes in the pathway are expressed in alternative combinations. These synthetic transcriptional programs, by combinatorially altering metabolic organization, allow us to flexibly redirect pathway product output between five distinct possible output states. Finally, we show that dCas9 can act as a master regulator of these gene expression programs, receiving input signals and acting as a single control point for the execution of a multi-gene response encompassing simultaneous activation and repression of downstream target genes.

CRISPR scaffold RNAs encode both target locus and regulatory function
scRNAs enable multi-gene transcription programs with simultaneous activation and repression
scRNAs function efficiently in human and yeast cells Simultaneous control of multiple genes enables flexible manipulation of a complex pathway

### Results

### CRISPR RNA Scaffolds Efficiently Activate Gene Expression in Yeast

The minimal sgRNA that has previously been used in CRISPR engineering consists of several modular domains: a 20 nucleotide variable DNA targeting sequence and two structured RNA domains—the dCas9-binding domain and a 3′ tracrRNA domain—which are necessary for proper structure formation and binding to Cas9 (Jinek et al., 2012; 2014; Nishimasu et al., 2014). Here, to generate scaffold RNA (scRNA) constructs with additional protein recruitment capabilities, we first introduced an additional single RNA hairpin domain to the 3′ end of the sgRNA, connected by a two base linker. For these recruitment RNA modules, we used the well-characterized viral RNA sequences MS2, PP7, and com, which are recognized by the MCP, PCP, and Com RNA binding proteins respectively. We fused the transcriptional activation domain VP64 to each of the corresponding RNA binding proteins.

We first tested the CRISPR scRNA platform in yeast. A strain containing a tet-promoter driven fluorescent protein reporter was transformed to express dCas9, modified scRNAs targeting the tet operator, and the corresponding VP64 fusion proteins. We observed significant reporter gene expression using each of the three tested RNA binding recruitment modules (FIG. 2A). scRNA constructs with recruitment hairpin domains connected to the sgRNA by linkers longer than two bases (up to 20 bases) gave weaker reporter gene expression (FIG. 7A). scRNA designs with recruitment sequences attached to the 5′ end of the sgRNA gave no significant activation and were not examined further.

Gene activation mediated by scRNA-recruitment of VP64 was substantially greater than that for the direct dCas9-VP64 fusion protein. Both MCP and PCP bind to their corresponding RNA targets as dimers (Chao et al., 2008), which may account for some of the difference. The oligomerization state of the Com protein has not been directly determined but functional data consistent with a Com monomer has been reported (Wulczyn and Kahmann, 1991).

### Three RNA-Protein Recruitment Modules Act in an Orthogonal Manner

To determine if there is any crosstalk between RNA hairpins and non-cognate binding proteins (e.g. MS2 RNA recruiting the PCP protein), we expressed all three RNA hairpin designs (MS2, PP7, and com) in yeast strains containing either the MCP, PCP, or Com fusion proteins. We used a 7×tetO reporter to ensure that we could observe any weak cross-activation. No significant crosstalk was detected between mismatched pairs of scRNA sequences and binding proteins (FIG. 2B). The strong activation of reporter gene expression only when cognate scRNA and RNA binding protein pairs are introduced demonstrates the potential for simultaneous, independent regulation of multiple target genes.

Multivalent Recruitment to scRNAs

To tune the valency of effectors recruited to each gene target, we introduced one, two, or three MS2 RNA hairpins to the 3′ end of the sgRNA. Surprisingly, reporter gene expression decreased with increasing numbers of MS2 hairpins (FIG. 7B). Northern blot analysis indicated that steady state RNA levels decreased with two or three MS2 hairpins, suggesting that RNA expression or stability is limiting for these constructs (FIG. 7C).

To address the apparent stability problem of multi-hairpin scRNAs, we constructed an alternative RNA design in which double-stranded linkers were inserted between the two repeats of the recruitment hairpins to enforce stable, local hairpin formation. These alternative designs produced stronger reporter gene activation for both MS2 and PP7 modules relative to the analogous single hairpin scRNAs (FIG. 2C). Northern blot analysis of the 2× constructs with double-stranded linkers indicated steady state RNA levels comparable to single hairpin scRNA and unmodified sgRNA constructs (FIG. 7C).

The strongest activation for a single scRNA construct was obtained by using a mixed hairpin construct containing two different recruitment motifs for the MCP-VP64 effector protein (2×MS2 (wt+f6))—this construct contained one MS2 hairpin and a second aptamer hairpin (f6) that had been selected to bind to the MCP protein (Hirao et al., 1998). Attempts to design 2× constructs with double-stranded linkers using the com RNA module were unsuccessful, possibly because the cognate Com protein binds to single stranded RNA at the base of the com hairpin (Hattman, 1999). RNA constructs with three MS2 hairpins connected by double-stranded linkers did not improve reporter gene expression beyond that obtained with the 2×MS2 scRNA. Northern blot analysis suggests that these constructs are stably expressed, so the lack of increased expression may be a result of misfolding or steric constraints.

To develop a platform for recruitment of more complex protein assemblies, we designed a heterologous MS2-PP7 scRNA sequence using the 2× double-stranded linker structure. Reporter gene activation was substantially stronger in yeast cells with both MCP-VP64 and PCP-VP64 effector proteins compared to cells with only a single type of effector protein, indicating that distinct RNA binding proteins can be recruited to the same target site (FIG. 2D). This provides an effective approach to combinatorially recruit multiple effectors for the logical control of target genes.

scRNAs can Mediate Activation of Reporter and Endogenous Genes in Human Cells

To test the efficacy of scRNA-based protein effector recruitment in human cells, we ported the system from yeast to HEK293 cells. The dCas9-binding hairpin of the sgRNA was modified as described previously to improve activity in human cells (see, e.g., (Chen et al., 2013). In HEK293 cells expressing dCas9, expression of an scRNA with the corresponding VP64 fusion protein effector produced substantial activation of a 7×tet-driven GFP reporter gene for all three RNA binding modules (FIG. 3A), although there are some quantitative differences from the activity trends observed in yeast. GFP activation with 1×MS2 and 1×PP7 scRNA constructs was relatively weak compared to both corresponding multivalent 2× scRNA constructs and the dCas9-VP64 fusion protein.

To determine if endogenous genes could be activated by targeting a single site upstream of the coding sequence, we designed 10 target sequences for the C-X-C chemokine receptor type 4 (CXCR4) (Table 3). CXCR4 expression is low in HEK293 cells, and changes in gene expression can be quantified at the single cell level by antibody staining. CXCR4 has previously been a target for CRISPR-based gene silencing in cell types with high basal expression levels (Gilbert et al., 2013). We used the divalent 2× (wt+f6) MS2 scRNA design to recruit the MCP-VP64 protein, and we observed increases in CXCR4 expression for nine of the ten target sites (FIG. 8). For the three strongest target sites, we compared CXCR4 activation mediated by scRNA to that with dCas9-VP64 and observed consistently stronger output with scRNA (FIG. 3B).

TABLE 3
Human sgRNA target sites used in this study.^{a}

| sgRNA | | Target | |
|---|---|---|---|
| target | DNA Sequence | Strand^{b} | Activity |
| sgTRE3G | GTACGTTCTCTATCACTGATA | NT | +++ |
| sgSV40.P1 | GCATACTTCTGCCTGCTGGGGAG | NT | +++ |
| | CCTG | | |
| sgSV40.NT1 | GAATAGCTCAGAGGCCGAGG | NT | +++ |
| sgCXCR4.1 | GGCTAGGAACGCGTCTCTCTG | NT | + |
| sgCXCR4.2 | GCCTGAAGACAGGTGGGAAGCGC | NT | + |
| sgCXCR4.3 | GAGCCGGACAGGACCTCCCAG | NT | ++ |
| sgCXCR4.4 | GCGGGTGGTCGGTAGTGAGTC | NT | +++ (C1) |
| sgCXCR4.5 | GGACCCTGCTGTTTGCGGGTGGT | NT | ++ |
| sgCXCR4.6 | GCAGACGCGAGGAAGGAGGGCGC | NT | +++ (C2) |
| sgCXCR4.7 | GCAAGTCACTCCCCTTCCCT | T | ++ |
| sgCXCR4.8 | GAATTCCATCCACTTTAGCAAGGA | T | + |
| sgCXCR4.9 | GCCCGCGCTTCCCACCTGTCTTC | T | − |
| sgCXCR4.10 | GCCTCTGGGAGGTCCTGTCCGGCT | T | +++ (C3) |
| | C | | |

^{a}If no 5′ G was present (required for expression from the U6 promoter), then a G was added to the target sequence. The TRE3G target site was selected as the only target sequence adjacent to an appropriate PAM motif (Qi et at., 2013) in the TRE3G promoter (Clonetech). The selected SV40 sites were described previously (Gilbert et at., 2013). 10 potential CXCR4
target sites were evaluated by antibody staining and FACS analysis. Sites 4, 6, and 10 gave the strongest expression, were redesignated C1, C2, and C3 respectively, and were used for further experiments (FIG. 3B).
^{b}Template strand (T) or non-template strand (NT).

scRNAs Recruit Chromatin Modifiers to Enhance Gene Silencing in Human Cells

In human cells, CRISPRi-mediated repression is relatively modest but can be enhanced by fusing dCas9 to the KRAB domain (Gilbert et al., 2013), a potent transcriptional repressor that recruits chromatin modifiers to silence target genes (Groner et al., 2010). To determine if scRNAs could recruit KRAB to enhance CRISPR-based gene silencing, we fused KRAB to RNA binding domains and designed scRNA constructs to target an SV40 promoter driving GFP expression. We targeted one site (P1) upstream of the transcriptional start site (TSS) and another site (NT1) that overlaps the TSS. Recruitment of a Com-KRAB fusion protein to either site by a com scRNA represses the GFP reporter beyond that obtained by CRISPRi alone (there is no significant CRISPRi effect at the P1 site upstream of the TSS) (FIG. 3C). The behavior of the KRAB domain recruited by scRNA was similar to that obtained with a direct dCas9-KRAB fusion protein. MCP-KRAB and PCP-KRAB fusion proteins were ineffective at mediating repression, potentially because MCP and PCP form dimers (Chao et al., 2008), which could interfere with KRAB function.

### Simultaneous On/Off Gene Regulation in Human Cells

The successful application of scRNA-mediated transcriptional control in human cells can provide simultaneous ON/OFF gene regulatory switches mediated by orthogonal RNA-binding proteins fused to transcriptional activators (VP64) or repressors (KRAB). To demonstrate this, we targeted endogenous CXCR4 for activation with MCP-VP64 while simultaneously targeting an additional endogenous gene for repression with COM-KRAB in HEK293T cells. We selected the β-1,4-N-acetyl-galactosaminyl transferase (B4GALNT1) gene from a set of target sites previously validated for repression with the dCas9-KRAB fusion protein (Gilbert et al., 2014). We observe simultaneous activation of CXCR4 and repression of B4GALNT1 measured by RT-qPCR, and these changes in gene expression are similar to that observed when single genes were targeted (FIG. 3D). In this experiment, activation and repression are mediated by a single scRNA for each target gene. Thus, this platform can be used for large-scale screening of pairwise combinations of genes that yield a target phenotype when one gene is activated and the other is repressed.

Harnessing scRNA Multi-Gene On/Off Transcriptional Programs to Redirect the Output of a Branched Metabolic Pathway in Yeast.

The complex multi-gene transcriptional programs that can be generated using scRNAs and dCas9 have the potential to rewire and control diverse cellular networks. One particularly interesting application is metabolic control. In many cases it would be very useful to synthetically reroute metabolic flux in biotechnology production strains, especially in the case of branched metabolic pathways where key intermediates can be routed down competing branches. There is often competition between branches required for cell growth versus production of the desired product. In these cases, being able to facilely control the expression of sets of metabolic enzymes, especially with bidirectional (ON/OFF) control, is essential to optimizing new flux patterns and, thereby, production of the desired product (Paddon et al., 2013; Ro et al., 2006). There is a notable lack of approaches to flexibly and dynamically increase the expression of enzymes in a desired pathway branch while simultaneously downregulating the expression of enzymes in a competing branch.

To test the ability of our scRNA programs to redirect metabolic pathway outputs, we turned to the highly-branched bacterial violacein biosynthetic pathway (Hoshino, 2011). The complete five-gene pathway (VioABEDC) produces the violet pigment violacein, and branch points at the last two enzymatic steps (VioD and VioC) can direct pathway output among four distinctly-colored products (FIG. 4A). The five-gene pathway can be reconstituted in yeast, and tuning the promoter strength for expression of VioD and VioC redirects pathway output to different products in a predictable manner (Lee et al., 2013). The four product states are visually distinguishable in yeast colonies and easily quantified by HPLC, making this pathway an ideal model system to simultaneously tune expression levels of multiple independent target genes to control functional output states.

We designed a yeast reporter strain with two key control points: the first control point (VioA) regulates total precursor flux into the pathway and the second control point regulates flow at the VioC/VioD branch point. The starting reporter strain has the VioBED genes under the control of strong promoters and VioAC genes under the control of weak promoters (FIG. 4B and Table 4), so that turning VioA ON will drive flux into the pathway, and flipping the ON/OFF expression states VioC and VioD genes will redirect the product output. The eight possible pairwise ON/OFF combinations of these three genes leads to five distinct output states: one state with complete pathway output off and four alternative product states when the pathway is on. To access all five states, we designed an scRNA program to target VioA and VioC with independent activators (2×PP7 and 1×MS2, respectively) and to target VioD with CRISPRi-mediated repression (FIG. 4B and Table 2). Activation of VioA in this reporter strain routes pathway flux to the proviolacein product (PV) (FIG. 4C). Once VioA is activated, activation of VioC or repression of VioD reroutes flux in a predictable manner. Expressing all three scRNA constructs simultaneously activates VioA and VioC and represses VioD to route flux into the pathway and to the deoxyviolacein (DV) product. Thus, in summary, the scRNA/dCas9 platform is highly flexible and efficient at generating all of the multi-gene transcriptional states necessary to yield all possible metabolic outputs of the violacein pathway.

TABLE 2
Yeast sgRNA target sites used in this study.^{a}

| sgRNA | | Target | |
|---|---|---|---|
| target | DNA Sequence | Strand^{b} | Activity |
| sgTET | ACTTTTCTCTATCACTGATA | NT | +++ |
| sgTEF | TTGATATTTAAGTTAATAAA | T | +++ |
| sgREV1.1 | ATATATAGAGTTAGAGTTTA | T | + |
| sgREV1.2 | CATCGCATCAACTTAAACAT | T | + |
| sgREV1.3 | AAGACGGAAAAAAGTAGCTA | T | +++ |
| sgREV1.4 | TTAGCTACTTTTTTCCGTCT | NT | ++ |
| sgREV1.5 | TGAATTGAATGCTTTGAGTT | T | − |
| sgREV1.6 | TTTTAATCTGGCTTACAGAT | NT | − |
| sgREV1.7 | TTTAAAGTGATTAAAATATG | NT | − |
| sgREV1.8 | TTAATCACTTTAAAATAAAA | T | − |
| sgRNR2.1 | TGAGAGAATGAGAGTTTTGT | T | − |
| sgRNR2.2 | ATAGCACCGTACCATACCCT | T | +++ |
| sgRNR2.3 | ATTTCGAGTTTCCAAGGGTA | NT | ++ |
| sgRNR2.4 | AAGCAAAGGAGGGGAAGCAC | T | ++ |
| sgRNR2.5 | GTGCTACGAAGTGGTGTCTG | NT | +++ |
| sgRNR2.6 | CGCAGGGAGGTCTGGGTGTG | NT | − |
| sgRNR2.7 | ACCCAGACCTCCCTGCGAGC | T | − |
| sgRNR2.8 | GGAGCAACGGGCAACCGTTT | T | − |

^{a}The selected TET and TEF target sites were described previously (Gilbert et at., 2013). sgTET was used for reporter gene activation experiments. sgTEF was used to silence expression from pTEF1-VioD. For activation of Vio pathway genes driven by REV1 (VioA) and RNR2 (VioC) promoters (see Table 4), 8 sites upstream of the transcriptional start site and adjacent to an appropriate PAM motif (Qi et at., 2013) were screened for each gene. Activity was evaluated by visual inspection of yeast color development. Rev1.3 and Rnr2.5 were used for subsequent experiments.
^{b}Template strand (T) or non-template strand (NT).

TABLE 4
Yeast strains used in this study.

| Strain | Description | Genotype |
|---|---|---|
| SO992 | W303 derivative | MATa ura3 leu2 trp1 his3 can1R |
| | | ade |
| cSLQ.sc002 | W303 rtTA-msn2 | SO992 HO::rtTA-msn2_hph^{R} |
| cSLQ.Sc003 | cSLQ.sc002 | cSLQ.Sc002 trp1::pTET07-Venus |
| | pTET07-Venus | |
| yJZC02 | cSLQ.sc002 | cSLQ.Sc002 trp1::pTET01-Venus |
| | pTET01-Venus | |
| BY4741 | S288C derivative | MATa ura3 leu2 his3 met15 |
| yML017^{a} | BY4741 Vio-ABEDc | BY4741 his3::pCCW12-VioA/ |
| | | pTdh3-VioB/pPGK1-VioE/ |
| | | pTEF1-VioD/pRNR2-VioC |
| yML025^{b} | BY4741 Vio-aBEDc | BY4741 his3::pRev1-VioA/ |
| | | pTdh3-VioB/pPGK1-VioE/ |
| | | pTEF1-VioD/pRNR2-VioC |

^{a}VioABED genes are driven by strong promoters. VioC is driven by the comparatively weak RNR2 promoter (Lee et al., 2013).
^{b}VioBED genes are driven by strong promoters. VioA and VioC are driven by the comparatively weak REV1 and RNR2 promoters (Lee et al., 2013).

dCas9 Acts as a Master Regulator to Execute a Complex RNA-Encoded Expression Program

The dCas9 protein is a central regulatory node in the execution of scRNA-mediated gene expression programs, raising the possibility that it could act as a single synthetic master regulator, controlling expression levels for multiple downstream genes (FIG. 5A). We designed a system in which expression of dCas9 controls a switch from a cell type that produces the PV metabolic product to one that produces DV. Expression of dCas9 was controlled by an inducible pGal10-dCas9 construct. The starting yeast strain contained the VioABED genes under the control of strong promoters, and VioC under the control of a weak promoter (Table 4). We introduced a two-scRNA program to switch VioC/VioD from OFF/ON to ON/OFF, redirecting output from PV to DV. When all components are present in yeast, but Gal inducer is absent, PV is the dominant product. However, when this strain is grown in the presence of Gal, dCas9 is expressed to execute the simultaneous switch of VioC to the ON state and VioD to the OFF state such that pathway output is routed to DV (FIG. 5B). Thus, multiple scRNAs can be regulated using expression of the dCas9 protein as a single control point.

### Discussion

### CRISPR Toolkit Enables Construction of Complex Regulatory Circuits

A wide range of CRISPR-related technologies have recently emerged for editing and manipulating target genomes (Mali et al., 2013b; Sander and Joung, 2014). A key advantage of these tools is that they interface with core biological mechanisms, thus allowing the system to be easily ported between different organisms. Watson-Crick base-pairing rules specify target site selection, and synthetic effector proteins interface with conserved features of the transcriptional machinery to control gene expression. Here we have expanded the scope of the CRISPR toolkit further by adding another basic feature of biological systems, spatial organization mediated by scaffolding molecules, to link functional effector domains to genomic target sites. A modular scaffold RNA encodes, within a single molecule, the information specifying the target site in the genome and the particular regulatory function to be executed at that site. scRNAs encode this information using a 5′ 20 base targeting sequence, a common dCas9-binding domain, and a 3′ protein recruitment domain. Expression of multiple RNA scaffolds simultaneously permits independent, programmable control of multiple genes in parallel. Most simply, this approach provides a straightforward method to implement simultaneous multi-gene ON/OFF regulatory switching programs.

scRNAs allow straightforward fine-tuning of output levels in a more analog fashion by altering the valency of effector proteins recruited to an individual target site. Although not explored here, an additional layer of expression control could come from the choice of scRNA target site. In this work we screened several candidate target sites to identify those that produced maximal output for further analysis (FIG. 8, Table 2 & 3). To access a range of intermediate output levels, target sites that are less effective could also be selected. More systematic screening approaches will provide general rules to select target sites for varying output levels (Gilbert, Horlbeck, Weissman et al., submitted).

Finally, there are many different classes of protein effectors and epigenetic modifiers that could be recruited via scRNAs to produce different levels and types of gene and pathway activation or repression. Although here we have only focused on the general regulatory categories of activation and repression, there are clearly more distinct, qualitatively different subclasses of regulation, including, for example, regulators that can produce stable, long-lived chromatin states that persist well after an input stimulus is removed. Recent progress towards recruiting a library of epigenetic modifiers with zinc finger proteins (Keung et al., 2014) suggests that a similar range of functionality could be achieved by recruitment via scRNAs. Thus it may be possible to construct even more nuanced and sophisticated gene expression programs by using a variety of regulators with CRISPR scRNAs, and by recruiting these regulators in a combinatorial fashion.

These scRNA-encoded transcriptional programs have several key advantages that are lacking in most transcriptional engineering platforms. First, they are easily programmable and parallel in that they rely on the simple design of scRNAs that use Watson-Crick base pairing to target desired endogenous loci in the genome. TAL effectors can be used to generate complex programs, but this requires the custom design of many distinct TAL specificities. Second, scRNA programs allow for distinct regulatory actions to take place at each targeted locus. While CRISPRi programs can be targeted to many distinct sites in the genome, fusing or tethering a regulatory effector directly to the Cas9 protein only allows one type of regulatory event (e.g. activation or repression) to take place at all of the targeted loci. By tethering effectors to binding motifs in the scRNA, which also encodes the loci targeting information, we have created single RNA molecules that modularly specify both a target loci and regulatory outcome in their sequence. Third, although the scRNA programs can involve many genes (based on how many scRNAs are expressed), they can still be controlled by a single master regulatory event—the expression of the dCas9 protein. Thus one still has temporal control over the entire multi-gene program.

Orthogonal dCas9 proteins from other species (besides S. pyogenes) can recognize guide RNAs with different dCas9 binding modules (Esvelt et al., 2013) and thus can provide another potential layer for modular control in CRISPR engineered transcriptional circuits that is complementary to the scaffold RNAs explored here (FIG. 6). For example, one can imagine creating, in one single cell, alternative sets of scRNA programs, each corresponding to an orthogonal dCas9 ortholog. In such a case, one could switch between distinct programs by controlling the expression of the dCas9 master regulators.

### Applications: Reprogramming Complex Networks Controlling Cell Function and Fate

These key features of scRNA encoded transcriptional programs can make them powerful tools for manipulating complex cellular behaviors, such as differentiation or metabolism. As explored here, such customized expression programs could be useful for metabolic engineering. Microorganisms can be engineered for the synthesis of desirable molecules by heterologous expression of the desired metabolic pathway. Designing these microbial production factories requires careful engineering to prevent detrimental effects on host growth and metabolism, to avoid buildup of toxic intermediates, and to coordinate the expression of multiple genes to switch from growth to production phase (Keasling, 2012). Often optimizing production requires the coordinated increase in the expression of enzymes that convert key branch point precursors into the desired product, as well as simultaneous repression of enzymes that deplete these precursors towards alternative products. Moreover, since these alternative products are often necessary for growth, optimized production requires precise and coordinated temporal control of when growth branches are repressed and production branches are activated. It is difficult to construct complex programs of this type with only a handful of well-characterized inducible promoters.

A CRISPR RNA-encoded gene expression program is ideally suited to address these challenges by activating multiple target pathway genes while simultaneously repressing multiple branch points that divert metabolites to cell growth. Execution of the program can be controlled by a dCas9 master regulator that is induced at the appropriate time to divert metabolites from growth to target molecule production. To avoid toxic intermediate buildup, expression levels of target pathway genes can be tuned to different levels, using differential multivalent recruitment of activators, to prevent bottlenecks.

To improve metabolite production, CRISPR RNA-based scaffolds could also be used as a rapid prototyping strategy to screen for gene expression programs that simultaneously alter the expression levels of multiple metabolic enzymes. scRNA libraries will allow screening of combinations of genes for up/down regulation. The regions of expression space that are then identified by such screens could then be custom constructed with specific promoters to achieve finer control. CRISPR tools can also be combined by other approaches to perturb and optimize metabolic gene networks. Global transcription machinery engineering (gTME) screens mutations in general transcription factors or coactivators to modify the expression of many genes simultaneously (Alper et al., 2006). gTME could be used to identify potential target genes for control by scRNA-encoded programs and a dCas9 master regulator. Alternatively, a dCas9 master regulator could be used to switch between global transcription programs by activating and repressing modified general transcription factors that elicit global changes in gene expression.

Finally scRNA/CRISPR programs are easily transferable to many different hosts. Most metabolic engineering efforts use well-characterized and genetically tractable hosts like E. coli or S. cerevisiae, but CRISPR-based tools to modify and regulate host genomes may dramatically expand the space of microorganisms that can be engineered for biosynthesis. Microbial strains or plants that have desirable industrial characteristics or metabolic precursors but lack good tools for genome manipulation may now be accessible for engineering. Instead of using heterologous hosts, it may even become routine to use CRISPR-based tools to optimize target molecule production in the native host organism for the desired pathway.

Another broad area of potential applications for such customized expression programs is in controlling cell fate decisions. During development, master regulators specify cell fates by directly or indirectly regulating multiple downstream target genes, and their presence or absence can determine the outcome of a developmental lineage (Chan and Kyba, 2013). A CRISPR-based multidirectional ON/OFF switch program could provide a straightforward method for genetic reprogramming by synthetically mimicking the behavior of master regulators. scRNA programs could be used to simultaneously activate and repress different master regulators, or to bypass master regulators and directly engage the next layer of target genes to specify cell fates. scRNA programs could also be used to create customized hybrid cell fate states that are not generated by natural master regulators, but that might still be useful in a therapeutic or research context. In either scenario, the ability of dCas9 itself to act as a synthetic master regulator will be a useful tool for controlling the timing of differentiation. Synthetic control of cell fate reprogramming could provide powerful new tools for regenerative medicine or other cell-based therapeutics.

### RNA Recruitment as a Discovery Tool for Biology

CRISPR-based RNA scaffolds for programmable gene expression provide new tools to interrogate complex biological processes. High-throughput synthetic lethal screens have proven extremely powerful in analyzing complex biological systems and shedding light on strategies for treating disease networks. Such screens, however, whether they utilize siRNAs or CRISPRi sgRNAs, rely on perturbing the expression of multiple genes in one direction (usually repression). It is equally likely that we can learn new features of networks by, in a high-throughput manner, simultaneously activating and repressing different combinations of genes. This is particularly true in cases in which a particular cellular outcome requires both activation of that response, but also simultaneous inactivation of genes involved in driving competing, alternative responses (Rais et al., 2013). The multi-directional, but high-throughput, regulation that can be achieved with the scRNA/CRISPR platform is ideal for this type of exploration.

### Experimental Procedures

scRNA Sequence Design

sgRNA sequences were extended to include hairpin sequences for MS2 (C5 variant) (Lowary and Uhlenbeck, 1987), PP7 (Lim et al., 2001), or com (Hattman, 1999). Sequences for linkers to the guide RNA and between hairpins were designed with RNA Designer (Andronescu et al., 2004). Candidate sequences were linked to the complete sgRNA sequence and evaluated in NUPACK (Zadeh et al., 2011) to confirm that the extended hairpins were compatible with sgRNA folding. Successful candidates were then evaluated for function in yeast as described below. The 2×MS2 (wt+f6) scRNA design uses the SELEX f6 aptamer, which was selected to bind the MCP protein (Hirao et al., 1998). Sequences of the minimal sgRNA, extended scRNAs, and RNA-binding modules are described in the Extended Experimental Procedures and Table 1.

TABLE 1
RNA binding modules for yeast scRNA constructs
used in this study.^{a}

| | RNA | |
|---|---|---|
| | Binding | |
| Plasmid | Module | DNA Sequence |
| pJZC545 | 1x MS2 | GCGCACATGAGGATCACCCATGTGC |
| pJZC583 | 2x MS2 | GGGAGCACATGAGGATCACCCATGTGCCACGAGC |
| | | GACATGAGGATCACCCATGTCGCTCGTGTTCCC |
| pJZC588 | 2x (wt + | GGGAGCACATGAGGATCACCCATGTGCGACTCCC |
| | f6) MS2 | ACAGTCACTGGGGAGTCTTCCC |
| pJZC548 | 1x PP7 | AACATAAGGAGTTTATATGGAAACCCTTATG |
| pJZC603 | 2x PP7 | GGGAGCTAAGGAGTTTATATGGAAACCCTTAGCC |
| | | TGCTGCGTAAGGAGTTTATATGGAAACCCTTACG |
| | | CAGCAGTTCCC |
| pJZC572 | 1x com | CTGAATGCCTGCGAGCATC |
| pJZC593 | MS2-PP7 | GGGAGCACATGAGGATCACCCATGTGCCACGAGT |
| | | AAGGAGTTTATATGGAAACCCTTACTCGTGTTCC |
| | | C |

^{a}To generate complete scRNA sequences with alternative RNA binding modules, replace the 1x MS2 sequences (See, extended experimental procedures) with the appropriate sequence from the table.

### Plasmid Design for CRISPR in Yeast

Mammalian codon-optimized S. pyogenes dCas9 (Qi et al., 2013) with three C-terminal SV40 NLSs was expressed from a constitutive Tdh3 or inducible Gal10 promoter. The dCas9-VP64 fusion protein was constructed with two C-terminal SV40 NLSs, the VP64 domain (Beerli et al., 1998), and an additional SV40 NLS. RNA-binding proteins MCP (ΔFG/V29I mutant) (Lim and Peabody, 1994), PCP (ΔFG mutant) (Chao et al., 2008), and Com (Hattman, 1999) were expressed with an N-terminal SV40 NLS and a C-terminal VP64 fusion domain. All protein expression constructs were integrated in single copy into the yeast genome. Complete descriptions of these constructs are provided in Table 5. sgRNA constructs were expressed from the pRS316 CEN/ARS plasmid (ura3 marker) with the SNR52 promoter and SUP4 terminator (DiCarlo et al., 2013). sgRNA target sites are listed in Table 2. 20 base guide sequences upstream of an appropriate PAM motif for S. pyogenes dCas9 (Qi et al., 2013) were selected. For target genes that had not been previously targeted for CRISPR-based transcriptional regulation, we screened 8 candidate target sites upstream of the gene and tested each site independently for the desired output (Table 2). The target site with the strongest effect on output was used for subsequent experiments.

TABLE 5
Yeast protein expression plasmids used in this study.

| | Parent | | Pro- | | Termi- |
|---|---|---|---|---|---|
| Plasmid^{a} | Vector^{b} | Marker | moter | Gene | nator^{b} |
| pJZC518 | pNH605 | leu2 | pTdh3 | dCas9 | C. alb. |
| | | | | | Adh1 |
| pJZC519 | pNH605 | leu2 | pTdh3 | dCas9-VP64 | C. alb. |
| | | | | | Adh1 |
| pJZC522 | pNH603 | his3 | pAdh | MCP-VP64 | C. alb. |
| | | | | | Adh1 |
| pJZC504 | pNH603 | his3 | pAdh | PCP-VP64 | C. alb. |
| | | | | | Adh1 |
| pJZC506 | pNH603 | his3 | pAdh | COM-VP64 | C. alb. |
| | | | | | Adh1 |
| pJZC620 | pNH605 | leu2 | 1) pAdh | 1) MCP-VP64 | 1) Eno2 |
| | | | 2) pAdh | 2) PCP-VP64 | 2) Adh2 |
| | | | 3) pTdh3 | 3) dCas9 | 3) C. alb. |
| | | | | | Adh1 |
| pJZC638 | pNH605 | leu2 | 1) pAdh | 1) MCP-VP64 | 1) Eno2 |
| | | | 2) pGal10 | 2) dCas9 | 2) C. alb. |
| | | | | | Adh1 |

^{a}Separate plasmids containing dCas9 and effector protein expression cassettes were used for all reporter gene experiments. Plasmids combining RNA-binding protein effectors and dCas9 in 2 or 3 gene cassettes (pJZC620 and 638) were used for violacein pathway experiments. Control experiments in reporter gene yeast strains gave indistinguishable results when protein expression cassettes were introduced individually at separate loci or together in a single plasmid.
^{b}The pNH600 series of yeast single copy integration vectors has been described previously (Zalatan et al., 2012).

### Yeast Strain Construction and Manipulation

Yeast (S. cerevisiae) transformations were performed with the standard lithium acetate method. The parent yeast strain for reporter gene experiments was SO992 (W303; MATa ura3 leu2 trp1 his3). Reporter strains were generated with genomic integrated TetON-Venus reporters and an rtTA-msn2 gene. TetON reporters were introduced with either 7× or 1× repeats of the tet operator sequence. The rtTA gene allows doxycycline induction of the tet reporter as a positive control. Complete descriptions of yeast strains are provided in Table 4. After transformations of CRISPR components, yeast strains were grown overnight at 30° C. in the appropriate media (SD complete or SD-Ura). Overnight cultures were diluted 1:50 and grown for an additional 4 hours. Fluorescent protein expression levels were measured with a LSRII flow cytometer (BD Biosciences).

### Yeast Violacein Production

Yeast strains for violacein biosynthesis were constructed and product distributions were analyzed as described previously (Lee et al., 2013) with minor modifications. The parent yeast strain for these experiments was BY4741 (S288C; MATa ura3 leu2 his3 met15). Complete 5-gene cassettes for violacein pathway production were integrated at the his3 locus. Strain yML025 contains strong promoters driving VioBED genes and weak promoters driving VioAC genes; strain yML017 contains strong promoters driving VioABED genes and a weak promoter driving VioC (Table 4). 2 or 3 gene cassettes containing RNA-binding protein effectors and dCas9 were integrated at leu2 (Table 4). sgRNA constructs were expressed from a pRS316 vector as described above (Table 6). To introduce 2 or 3 sgRNA constructs simultaneously, multiple promoter-sgRNA-terminator cassettes were cloned together in a single plasmid using the In-Fusion method (Clonetech). Yeast strains with violacein pathway genes and the CRISPR system with constitutive dCas9 expression were grown on SD-Ura agar plates. Strains with gal-inducible dCas9 were grown on SD-Ura (Gal OFF) or SSG-Ura (synthetic media/2% sucrose/2% galactose, Gal ON). After 3 days at 30° C., approximately 12 mg of yeast cells were harvested from plates, suspended in 250 μL methanol and boiled at 95° C. for 15 minutes, vortexing twice during the incubation. Solutions were centrifuged twice to remove cell debris, and the supernatant (extract) was analyzed by HPLC on an Agilent Rapid Resolution SB-C18 column as described previously (Lee et al., 2013).

TABLE 6
Yeast sgRNA expression plasmids for violacein pathway targets

| Plasmid | Target Gene | Target Site | RNA Design |
|---|---|---|---|
| pJZC603 | pREV1-VioA | REV1.3 | 2x PP7 |
| pJZC639 | 1) pREV1-VioA | 1) REV1.3 | 1) 2x PP7 |
| | 2) pRNR2-VioC | 2) RNR2.5 | 2) 1x MS2 |
| pJZC640 | 1) pREV1-VioA | 1) REV1.3 | 1) 2x PP7 |
| | 2) TEF1-VioD | 2) TEF | 2) sgRNA |
| pJZC641 | 1) pREV1-VioA | 1) REV1.3 | 1) 2x PP7 |
| | 2) pRNR2-VioC | 2) RNR2.5 | 2) 1x MS2 |
| | 3) TEF1-VioD | 3) TEF | 3) sgRNA |
| pJZC642 | 1) TEF1-VioD | 1) TEF | 1) sgRNA |
| | 2) pRNR2-VioC | 2) RNR2.5 | 2) 1x MS2 |

^{a} sgRNA constructs were expressed from the pRS316 CEN/ARS plasmid with the SNR52 promoter and a SUP4 terminator (DiCarlo et al., 2013). The selection marker is ura3.

### Northern Blotting

Yeast strains containing sgRNA expression cassettes were grown in SD-Ura. Total RNA was extracted as described (Kagansky et al., 2009). 10 μg of total RNA samples were electrophoresed on Novex 6% TBE-Urea PAGE gels (Life Technologies) in 0.5×TBE buffer at 150V, transferred to Hybond NX membranes (GE Healthcare) in 0.5×TBE for 1.5 hours at 250 mA using a Mini Protean Tetra Cell apparatus (Bio-Rad) and UV crosslinked on a Stratalinker (Stratagene, 2×120 μJ/cm²). The membranes were probed with a 5′-³²P-labeled DNA oligonucleotide 5′-TTGATAACGGACTAGCCTTAT (FIG. 7) diluted in modified Church-Gilbert buffer (0.5 M phosphate pH 7.2, 7% (w/v) SDS, 10 mM EDTA) with overnight incubation at 42° C. Blots were washed 3× for 20 min at 50° C. in 2×SSC, 0.2% SDS before mounting for exposure with a storage phosphoscreen (GE Healthcare). Images were obtained on a Typhoon 9410 scanner (GE Healthcare) after exposure durations of 4 h to overnight. A negative control yeast strain lacking the sgRNA expression cassette gave no detectable probe hybridization.

### Plasmid Design for CRISPR in Human Cells

Plasmids for expression of S. pyogenes dCas9, dCas9 fusion proteins, and sgRNA constructs were described previously (Gilbert et al., 2013). dCas9 constructs were expressed from an SFFV promoter with two C-terminal SV40 NLSs and a tagBFP. The dCas9-KRAB fusion protein was constructed with a KRAB domain (Margolin et al., 1994) fused to the C-terminus of the tagBFP. The dCas9-VP64 fusion protein was constructed with two C-terminal SV40 NLSs, the VP64 domain, an additional SV40 NLS, and a tagBFP. sgRNA sequences were modified as described previously for expression in human cells (see, e.g., (Chen et al., 2013). sgRNAs were expressed using a lentiviral U6-based expression vector derived from pSico that expresses mCherry from a CMV promoter. To simultaneously express sgRNAs and RNA-binding protein effectors, the mCherry cassette was modified to express the protein effector followed by an IRES and mCherry. RNA-binding proteins (MCP, PCP, and Com) were expressed with an N-terminal SV40 NLS and a C-terminal VP64 or KRAB fusion domain. Complete descriptions of these constructs are provided in Table 7. sgRNA target site sequences are listed in Table 3. For human gene targets, guide sequences of 20-25 bases upstream of a PAM motif were selected. If no 5′ G was present (required for expression from U6), then a G was added to the sequence. sgRNA target sites for SV40-GFP were described previously (Gilbert et al., 2013).

TABLE 7
Human plasmids for simultaneous expression
of scRNA and protein effectors.^{a}

| Plasmid | RNA Target | RNA Design | Protein Effector |
|---|---|---|---|
| pJZC35 | TRE3G | sgRNA | — |
| pJZC32 | TRE3G | sgRNA | MCP-VP64 |
| pJZC25 | TRE3G | 1x MS2 | MCP-VP64 |
| pJZC33 | TRE3G | 2x MS2 | MCP-VP64 |
| pJZC34 | TRE3G | 2x (wt + f6) MS2 | MCP-VP64 |
| pJZC41 | TRE3G | sgRNA | PCP-VP64 |
| pJZC39 | TRE3G | 1x PPV | PCP-VP64 |
| pJZC40 | TRE3G | 2x PP7 | PCP-VP64 |
| pJZC101 | TRE3G | sgRNA | Com-VP64 |
| pJZC48 | TRE3G | 1x com | Com-VP64 |
| pJZC102 | SV40.P1 | sgRNA | — |
| pJZC77 | SV40.P1 | sgRNA | Com-KRAB |
| pJZC78 | SV40.P1 | 1x com | Com-KRAB |
| pJZC103 | SV40.NT1 | sgRNA | — |
| pJZC73 | SV40.NT1 | sgRNA | Com-VP64 |
| pJZC74 | SV40.NT1 | 1x com | Com-VP64 |

^{a}Plasmids were derived from pSico with a U6 promoter to express RNA. A CMV promoter drives protein expression, followed by an IRES sequence and mCherry.

### Cell Culture, DNA Transfections, Viral Production, and Fluorescence Measurements in Human Cells

HEK293 cells were maintained in Dulbecco's modified Eagle medium (DMEM) in 10% FBS. Lentivirus was produced by transfecting HEK293 cells with standard packaging vectors. Pure populations of stable cell lines were sorted by flow cytometry using a BD FACS Aria2. Stable, sorted HEK293 cells lines expressing EGFP from an SV40 promoter and dCas9 or dCas9-KRAB were described previously (Gilbert et al., 2013). An HEK293 cell line with a TRE3G-EGFP reporter (Clonetech) was generated by lentiviral infection, transiently transfected with an rtTA transactivator protein, stimulated with doxycycline, and sorted for GFP expression. dCas9 or dCas9-VP64 were introduced by lentiviral infection and sorted for BFP expression. scRNA/protein effector cassettes were introduced into stable cell lines by lentiviral infection. For TRE3G-EGFP reporter gene activation experiments, cells were harvested on day 3 for FACS analysis. For SV40-EGFP reporter gene repression experiments, cells were split at day 3 and harvested on day 6. Cells were trypsinized to a single cell suspension and gated on the mCherry-positive population. For CXCR4 gene activation, cells on day 3 were dissociated in Gibco Cell Dissociation Buffer (PBS) and then stained in PBS/10% FBS for 1 hour at room temperature using an APC-coupled anti-human CXCR4 antibody (Biolegend) at 2 μg/mL. All flow cytometry analysis was performed using a LSR II flow cytometer (BD Biosciences).

### Extended Experimental Procedures

### Yeast Scaffold RNA Sequence Designs

scRNA sequences with RNA recruitment hairpins were constructed following the sgRNA sequence described previously (Qi et al., 2013). Unmodified sgRNA for CRISPRi in yeast were designed following (DiCarlo et al., 2013)—this sequence has a 3 base GGT extension of the 3′ tracr RNA.

Parent sgRNA
ACTTTTCTCTATCACTGATAGTTTTAGAGCTAGAAATAGCAAGTTAAAAT
AAGGCTAGTCCGTTATCAACTTGAAAAAGTGGCACCGAGTCGGTGGTGCT
TTTTTTGTTTTTTATGTCT
1x MS2 scRNA
ACTTTTCTCTATCACTGATAGTTTTAGAGCTAGAAATAGCAAGTTAAAAT
AAGGCTAGTCCGTTATCAACTTGAAAAAGTGGCACCGAGTCGGTGCGCGC
ACATGAGGATCACCCATGTGCTTTTTTTGTTTTTTATGTCT

Annotations: 20 base target site (TET), 1×MS2, SUP4 terminator

### Human Scaffold RNA Sequence Designs

The sgRNA sequence was modified for human cells as described (Chen et al., 2013) to remove a potential premature T₄ termination sequence and to extend the dCas9-binding hairpin. These changes had no detectable effect on function in yeast cells.

Parent sgRNA
GTACGTTCTCTATCACTGATAGTTTAAGAGCTATGCTGGAAACAGCATAG
CAAGTTTAAATAAGGCTAGTCCGTTATCAACTTGAAAAAGTGGCACCGAG
TCGGTGCTTTTTTT
1x MS2 scRNA
GTACGTTCTCTATCACTGATAGTTTAAGAGCTATGCTGGAAACAGCATAG
CAAGTTTAAATAAGGCTAGTCCGTTATCAACTTGAAAAAGTGGCACCGAG
TCGGTGCGCGCACATGAGGATCACCCATGTGCTTTTTTTGTTTTTTATGT
CT

Annotations: 20 base target site (TRE3G), 1×MS2, Tₙ terminator

### REFERENCES

Alper, H., Moxley, J., Nevoigt, E., Fink, G. R., and Stephanopoulos, G. (2006). Engineering yeast transcription machinery for improved ethanol tolerance and production. Science 314, 1565-1568.
Andronescu, M., Fejes, A. P., Hutter, F., Hoos, H. H., and Condon, A. (2004). A new algorithm for RNA secondary structure design. J. Mol. Biol. 336, 607-624.
Beerli, R. R., Segal, D. J., Dreier, B., and Barbas, C. F. (1998). Toward controlling gene expression at will: specific regulation of the erbB-2/HER-2 promoter by using polydactyl zinc finger proteins constructed from modular building blocks. P Natl Acad Sci Usa 95, 14628-14633.
Braglia, P., Percudani, R., and Dieci, G. (2005). Sequence context effects on oligo(dT) termination signal recognition by Saccharomyces cerevisiae RNA polymerase III. J. Biol. Chem. 280, 19551-19562.
Chan, S. S.-K., and Kyba, M. (2013). What is a Master Regulator? J Stem Cell Res Ther 3.
Chao, J. A., Patskovsky, Y., Almo, S. C., and Singer, R. H. (2008). Structural basis for the coevolution of a viral RNA-protein complex. Nat. Struct. Mol. Biol. 15, 103-105.
Chen, B., Gilbert, L. A., Cimini, B. A., Schnitzbauer, J., Zhang, W., Li, G.-W., Park, J., Blackburn, E. H., Weissman, J. S., Qi, L. S., et al. (2013). Dynamic imaging of genomic loci in living human cells by an optimized CRISPR/Cas system. Cell 155, 1479-1491.
Delebecque, C. J., Lindner, A. B., Silver, P. A., and Aldaye, F. A. (2011). Organization of intracellular reactions with rationally designed RNA assemblies. Science 333, 470-474.
DiCarlo, J. E., Norville, J. E., Mali, P., Rios, X., Aach, J., and Church, G. M. (2013). Genome engineering in Saccharomyces cerevisiae using CRISPR-Cas systems. Nucleic Acids Research 41, 4336-4343.
Esvelt, K. M., Mali, P., Braff, J. L., Moosburner, M., Yaung, S. J., and Church, G. M. (2013). Orthogonal Cas9 proteins for RNA-guided gene regulation and editing. Nat. Methods 10, 1116-1121.
Gaj, T., Gersbach, C. A., and Barbas, C. F. (2013). ZFN, TALEN, and CRISPR/Cas-based methods for genome engineering. Trends Biotechnol. 31, 397-405.
Gilbert, L. A., Larson, M. H., Morsut, L., Liu, Z., Brar, G. A., Torres, S. E., Stern-Ginossar, N., Brandman, O., Whitehead, E. H., Doudna, J. A., et al. (2013). CRISPR-mediated modular RNA-guided regulation of transcription in eukaryotes. Cell 154, 442-451.
Good, M. C., Zalatan, J. G., and Lim, W. A. (2011). Scaffold proteins: hubs for controlling the flow of cellular information. Science 332, 680-686.
Groner, A. C., Meylan, S., Ciuffi, A., Zangger, N., Ambrosini, G., Dénervaud, N., Bucher, P., and Trono, D. (2010). KRAB-zinc finger proteins and KAP1 can mediate long-range transcriptional repression through heterochromatin spreading. PLoS Genet 6, e1000869.
Hattman, S. (1999). Unusual transcriptional and translational regulation of the bacteriophage Mu mom operon. Pharmacol. Ther. 84, 367-388.
Hirao, I., Spingola, M., Peabody, D., and Ellington, A. D. (1998). The limits of specificity: an experimental analysis with RNA aptamers to MS2 coat protein variants. Mol. Divers. 4, 75-89.
Hoshino, T. (2011). Violacein and related tryptophan metabolites produced by Chromobacterium violaceum: biosynthetic mechanism and pathway for construction of violacein core. Appl. Microbiol. Biotechnol. 91, 1463-1475.
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-821.
Jinek, M., Jiang, F., Taylor, D. W., Sternberg, S. H., Kaya, E., Ma, E., Anders, C., Hauer, M., Zhou, K., Lin, S., et al. (2014). Structures of Cas9 endonucleases reveal RNA-mediated conformational activation. Science 343, 1247997.
Kagansky, A., Folco, H. D., Almeida, R., Pidoux, A. L., Boukaba, A., Simmer, F., Urano, T., Hamilton, G. L., and Allshire, R. C. (2009). Synthetic heterochromatin bypasses RNAi and centromeric repeats to establish functional centromeres. Science 324, 1716-1719.
Keasling, J. D. (2012). Synthetic biology and the development of tools for metabolic engineering. Metab. Eng. 14, 189-195.
Keung, A. J., Bashor, C. J., Kiriakov, S., Collins, J. J., and Khalil, A. S. (2014). Using targeted chromatin regulators to engineer combinatorial and spatial transcriptional regulation. Cell 158, 110-120.
Lee, M. E., Aswani, A., Han, A. S., Tomlin, C. J., and Dueber, J. E. (2013). Expression-level optimization of a multi-enzyme pathway in the absence of a high-throughput assay. Nucleic Acids Research 41, 10668-10678.
Lim, F., and Peabody, D. S. (1994). Mutations that increase the affinity of a translational repressor for RNA. Nucleic Acids Research 22, 3748-3752.
Lim, F., Downey, T. P., and Peabody, D. S. (2001). Translational repression and specific RNA binding by the coat protein of the Pseudomonas phage PP7. J. Biol. Chem. 276, 22507-22513.
Lowary, P. T., and Uhlenbeck, O. C. (1987). An RNA mutation that increases the affinity of an RNA-protein interaction. Nucleic Acids Research 15, 10483-10493.
Mali, P., Aach, J., Stranges, P. B., Esvelt, K. M., Moosburner, M., Kosuri, S., Yang, L., and Church, G. M. (2013a). CAS9 transcriptional activators for target specificity screening and paired nickases for cooperative genome engineering. Nat Biotechnol 31, 833-838.
Mali, P., Esvelt, K. M., and Church, G. M. (2013b). Cas9 as a versatile tool for engineering biology. Nat. Methods 10, 957-963.
Margolin, J. F., Friedman, J. R., Meyer, W. K., Vissing, H., Thiesen, H. J., and Rauscher, F. J. (1994). Krüppel-associated boxes are potent transcriptional repression domains. P Natl Acad Sci Usa 91, 4509-4513.
Nishimasu, H., Ran, F. A., Hsu, P. D., Konermann, S., Shehata, S. I., Dohmae, N., Ishitani, R., Zhang, F., and Nureki, O. (2014). Crystal structure of Cas9 in complex with guide RNA and target DNA. Cell 156, 935-949.
Paddon, C. J., Westfall, P. J., Pitera, D. J., Benjamin, K., Fisher, K., McPhee, D., Leavell, M. D., Tai, A., Main, A., Eng, D., et al. (2013). High-level semi-synthetic production of the potent antimalarial artemisinin. Nature 496, 528-532.
Qi, L. S., Larson, M. H., Gilbert, L. A., Doudna, J. A., Weissman, J. S., Arkin, A. P., and Lim, W. A. (2013). Repurposing CRISPR as an RNA-guided platform for sequence-specific control of gene expression. Cell 152, 1173-1183.
Rais, Y., Zviran, A., Geula, S., Gafni, O., Chomsky, E., Viukov, S., Mansour, A. A., Caspi, I., Krupalnik, V., Zerbib, M., et al. (2013). Deterministic direct reprogramming of somatic cells to pluripotency. Nature 502, 65-70.
Rinn, J. L., and Chang, H. Y. (2012). Genome regulation by long noncoding RNAs. Annu. Rev. Biochem. 81, 145-166.
Ro, D.-K., Paradise, E. M., Ouellet, M., Fisher, K. J., Newman, K. L., Ndungu, J. M., Ho, K. A., Eachus, R. A., Ham, T. S., Kirby, J., et al. (2006). Production of the antimalarial drug precursor artemisinic acid in engineered yeast. Nature 440, 940-943.
Sander, J. D., and Joung, J. K. (2014). CRISPR-Cas systems for editing, regulating and targeting genomes. Nat Biotechnol 32, 347-355.
Spitale, R. C., Tsai, M.-C., and Chang, H. Y. (2011). RNA templating the epigenome: long noncoding RNAs as molecular scaffolds. Epigenetics 6, 539-543.
Wulczyn, F. G., and Kahmann, R. (1991). Translational stimulation: RNA sequence and structure requirements for binding of Com protein. Cell 65, 259-269.
Zadeh, J. N., Steenberg, C. D., Bois, J. S., Wolfe, B. R., Pierce, M. B., Khan, A. R., Dirks, R. M., and Pierce, N. A. (2011). NUPACK: Analysis and design of nucleic acid systems. J. Comput. Chem. 32, 170-173.
Zalatan, J. G., Coyle, S. M., Rajan, S., Sidhu, S. S., and Lim, W. A. (2012). Conformational control of the Ste5 scaffold protein insulates against MAP kinase misactivation. Science 337, 1218-1222.

INFORMAL SEQUENCE LISTING

SEQ ID NO: 1: encodes Cas9 binding region opti-
mized for yeast
GTTTTAGAGCTAGAAATAGCAAGTTAAAATAAGGCTAGTCCGTTATCAAC
TTGAAAAAGTGGCACCGAGTCGGTGC
SEQ ID NO: 2: MCP polypeptide sequence
MASNFTQFVLVDNGGTGDVTVAPSNFANGIAEWISSNSRSQAYKVTCSVR
QSSAQNRKYTIKVEVPKGAWRSYLNMELTIPIFATNSDCELIVKAMQGLL
KDGNPIPSAIAANSGIY
SEQ ID NO: 3: PCP polypeptide sequence
MSKTIVLSVGEATRTLTEIQSTADRQIFEEKVGPLVGRLRLTASLRQNGA
KTAYRVNLKLDQADVVDSGLPKVRYTQVWSHDVTIVANSTEASRKSLYDL
TKSLVATSQVEDLVVNLVPLGR
SEQ ID NO: 4: COM polypeptide sequence
MKSIRCKNCNKLLFKADSFDHIEIRCPRCKRHIIMLNACEHPTEKHCGKR
EKITHSDETVRY
SEQ ID NO: 5: encodes ms2 sequence
GCGCACATGAGGATCACCCATGTGC
SEQ ID NO: 6: encodes f6 sequence
CCACAGTCACTGGG
SEQ ID NO: 7: encodes PP7 sequence
AACATAAGGAGTTTATATGGAAACCCTTATG
SEQ ID NO: 8: encodes coin sequence
CTGAATGCCTGCGAGCATC
SEQ ID NO: 9: encodes ms2-2Xds
GGGAGCACATGAGGATCACCCATGTGCCACGAGCGACATGAGGATCACCC
ATGTCGCTCGTGTTCCC
SEQ ID NO: 10: encodes ms2-2Xds-f6
GGGAGCACATGAGGATCACCCATGTGCGACTCCCACAGTCACTGGGGAGT
CTTCCC
SEQ ID NO: 11: encodes PP7-2Xds
GGGAGCTAAGGAGTTTATATGGAAACCCTTAGCCTGCTGCGTAAGGAGTT
TATATGGAAACCCTTACGCAGCAGTTCCC
SEQ ID NO: 12: encodes ms2-2Xds-PP7
GGGAGCACATGAGGATCACCCATGTGCCACGAGTAAGGAGTTTATATGGA
AACCCTTACTCGTGTTCCC
SEQ ID NO: 13: encodes Cas9 binding region opti-
mized for mammalian (e.g., human cells)
GTTTAAGAGCTATGCTGGAAACAGCATAGCAAGTTTAAATAAGGCTAGTC
CGTTATCAACTTGAAAAAGTGGCACCGAGTCGGTGC
SEQ ID NO: 14: seven consecutive uracils
TTTTTTT
SEQ ID NO: 15: SUP4 terminator
TTTTTTTGTTTTTTATGTCT
SEQ ID NO: 16: human ribosomal protein L7a (NP_
000963)
MPKGKKAKGK KVAPAPAVVK KQEAKKVVNP LFEKRPKNFG
IGQDIQPKRD LTRFVKWPRY IRLQRQRAIL YKRLKVPPAI
NQFTQALDRQ TATQLLKLAH KYRPETKQEK KQRLLARAEK
KAAGKGDVPT KRPPVLRAGV NTVTTLVENK KAQLVVIAHD
VDPIELVVFL PALCRKMGVP YCIIKGKARL GRLVHRKTCT
TVAFTQVNSE DKGALAKLVE AIRTNYNDRY DEIRRHWGGN
VLGPKSVARI AKLEKAKAKE LATKLG
SEQ ID NO: 17: human ribosomal protein L7a subunit
RNAB1
TRFVKWPRY IRLQRQRAIL YKRLKVPPAI NQFTQALDRQ
TATQLLKLAH
SEQ ID NO: 17: human ribosomal protein L7a subunit
RNAB2
KYRPETKQEK KQRLLARAEK KAAGKGDVPT KRPPVLRAGV
NTVTTLVENK KAQLVVIAHD V

1. A scaffold RNA (scRNA), wherein the scaffold RNA comprises:
a nucleic acid binding region, the nucleic acid binding region having a length of between about 15 to about 30 nucleotides, wherein the nucleic acid binding region is complementary to a target nucleic acid;
a 5′ scaffold region, wherein the 5′ scaffold region is 5′ of a 3′ scaffold region and specifically binds to at least one 5′ scaffold region binding polypeptide or small molecule;
the 3′ scaffold region, wherein the 3′ scaffold region is 3′ of the 5′ scaffold region and specifically binds to at least one 3′ scaffold region binding polypeptide or small molecule; and
a transcription termination sequence,
wherein the scaffold RNA is configured to recruit 5′ and 3′ scaffold region binding polypeptides or small molecules to the target nucleic acid.

2. The scRNA of claim 1, wherein the 5′ scaffold region and/or the 3′ scaffold region comprises one, two, or more RNA hairpins.

3. (canceled)

4. The scRNA of claim 1, wherein the 5′ scaffold region is 5′ or 3′ of the binding region.

5. (canceled)

6. (canceled)

7. The scRNA of claim 1, wherein the binding of a small molecule or polypeptide to the 5′ scaffold region and/or the 3′ scaffold region mediates the activity of the scRNA; and wherein the small molecule has a molecular weight of less than about 5,000; less than about 1,000; or less than about 500 daltons.

8. The scRNA of claim 1, wherein the binding of a small molecule to the 5′ scaffold region and/or the 3′ scaffold region mediates the binding of a polypeptide to the 5′ scaffold region and/or the 3′ scaffold region.

9. The scRNA of claim 7, wherein the activity of the scRNA comprises transcriptional modulation, chromatin modification, or target genetic element binding.

10. The scRNA of claim 1, wherein the 5′ scaffold region and/or the 3′ scaffold region is configured to bind a small guide RNA-mediated nuclease, and wherein the scaffold region configured to bind the small guide RNA-mediated nuclease is 3′ of the nucleic acid binding region.

11. The scRNA of claim 10, wherein the 5′ scaffold region and/or the 3′ scaffold region that is configured to bind a small guide RNA-mediated nuclease comprises an RNA sequence encoded by SEQ ID NO:1 or SEQ ID NO:13.

12. (canceled)

13. The scRNA of claim 1, wherein the 5′ scaffold region and/or the 3′ scaffold region is configured to bind one or more, or two or more, polypeptides, wherein at least one of the polypeptides comprises a transcriptional modulator or restriction endonuclease and an affinity domain having affinity for the 5′ scaffold region or the 3′ scaffold region.

14. The scRNA of claim 1, wherein the 5′ scaffold region and/or the 3′ scaffold region each comprises an ms2, f6, PP7, com, or L7a ligand sequence, wherein:
the ms2 sequence is configured to bind an MCP polypeptide or fragment thereof;
the f6 sequence is configured to bind an MCP polypeptide or fragment thereof;
the PP7 sequence is configured to bind a PCP polypeptide or fragment thereof;
the com sequence is configured to bind a COM polypeptide or fragment thereof; and
the L7a ligand sequence is configured to bind an L7a polypeptide or fragment thereof.

15. (canceled)

16. The scRNA of claim 14, wherein the ms2 sequence comprises or consists of an RNA sequence encoded by SEQ ID NO:5, the f6 sequence comprises or consists of an RNA sequence encoded by SEQ ID NO:6, the PP7 sequence comprises or consists of an RNA sequence encoded by SEQ ID NO:7, the com sequence comprises or consists of an RNA sequence encoded by SEQ ID NO:8, and the L7a ligand sequence comprises or consists of 30 consecutive riboguanine nucleotides.

17. The scRNA of claim 14, wherein the 5′ scaffold region and/or the 3′ scaffold region comprises or consists of one or more RNA sequences encoded by SEQ ID NO:9, SEQ ID NO:10, SEQ ID NO:11, or SEQ ID NO:12.

18. The scRNA of claim 13, wherein the transcriptional modulator comprises a transcriptional activator, a transcriptional repressor, or a chromatin modifier.

19. The scRNA of claim 18, wherein the transcriptional activator is VP16 or VP64, the transcriptional repressor is a KRAB domain, and the chromatin modifier is an enzyme that methylates, demethylates, acetylates or deacetylates histones.

20-24. (canceled)

25. An expression cassette comprising a heterologous promoter operably linked to a polynucleotide encoding a scRNA of claim 1.

26. (canceled)

27. A method for modulating transcription of a first target nucleic acid comprising:
contacting the first target nucleic acid with a first scRNA of claim 1, wherein the first scRNA binds to the first target nucleic acid;
or contacting a cell or cell extract containing the first target nucleic acid with a first expression cassette comprising a heterologous promoter operably linked to a polynucleotide encoding the first scRNA,
thereby modulating the transcription of the first target nucleic acid.

28. The method of claim 27, wherein the method further comprises contacting the target nucleic acid with a small guide RNA-mediated nuclease or contacting the cell or cell extract with an expression cassette containing a heterologous promoter operably linked to a polynucleotide encoding a small guide RNA-mediated nuclease.

29. The method of claim 27, wherein the method further comprises:
contacting a second target nucleic acid with a second structurally different scRNA of claim 1, wherein the second scRNA binds to the second target nucleic acid; or
contacting the cell or cell extract, wherein the cell or cell extract contain the first and second target nucleic acid, with a second structurally different expression cassette comprising a heterologous promoter operably linked to a polynucleotide encoding the second scRNA,
thereby modulating the transcription of the first and second target nucleic acids.

30. The method of claim 29, wherein the first scRNA activates or represses transcription of the first target nucleic acid and the second scRNA activates or represses transcription of the second target nucleic acid, and wherein the first and second scRNAs exhibit substantially no, or no, cross-talk.

31-33. (canceled)

34. A kit comprising a first and a second expression cassette, wherein:
the first expression cassette comprises a promoter operably linked to a polynucleotide containing a cloning region and a scaffold RNA framework, wherein the scaffold RNA framework comprises:
a 5′ scaffold region, wherein the 5′ scaffold region is 5′ of a 3′ scaffold region and specifically binds to at least one 5′ scaffold region binding polypeptide or small molecule;
the 3′ scaffold region, wherein the 3′ scaffold region is 3′ of the 5′ scaffold region and specifically binds to at least one 3′ scaffold region binding polypeptide or small molecule; and
a transcription termination sequence; and
the second expression cassette comprises a promoter operably linked to a small-guide RNA-mediated nuclease.

35. The kit of claim 34, wherein the 5′ scaffold region and/or the 3′ scaffold region comprises one, two, or more hairpins.

36. (canceled)

37. The kit of claim 34, wherein the 5′ scaffold region and/or the 3′ scaffold region is configured to bind a small guide RNA-mediated nuclease.

38. The kit of claim 37, wherein the 5′ scaffold region and/or the 3′ scaffold region that is configured to bind a small guide RNA-mediated nuclease comprises an RNA sequence encoded by SEQ ID NO:1 or SEQ ID NO:13.

39. (canceled)

40. The kit of claim 34, wherein the 5′ scaffold region and/or the 3′ scaffold region is configured to bind one or more, or two or more, polypeptides, and wherein at least one of the polypeptides comprises a transcriptional modulator and an affinity domain having affinity for the 5′ scaffold region or the 3′ scaffold region.

41. The kit of claim 34, wherein the 5′ scaffold region and/or the 3′ scaffold region comprises one or more ms2, f6, PP7, com, or L7a ligand sequences wherein:
the ms2 sequence is configured to bind an MCP polypeptide or fragment thereof;
the f6 sequence is configured to bind an MCP polypeptide or fragment thereof;
the PP7 sequence is configured to bind a PCP polypeptide or fragment thereof;
the com sequence is configured to bind a COM polypeptide or fragment thereof; and
the L7a ligand sequence is configured to bind an L7a polypeptide or fragment thereof.


SEQUENCE LISTING (55 sequences)

SEQ ID NO: 1 - DNA, 76 bp, Artificial Sequence
  synthetic nucleotide sequence
gttttagagctagaaatagcaagttaaaataaggctagtccgttatcaacttgaaaaagtggcaccgagtcggtgc

SEQ ID NO: 2 - protein, 117 aa, Artificial Sequence
  synthetic peptide construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 3 - protein, 122 aa, Artificial Sequence
  synthetic peptide construct
MSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 4 - protein, 62 aa, Artificial Sequence
  synthetic peptide construct
MKSIRCKNCNKLLFKADSFDHIEIRCPRCKRHIIMLNACEHPTEKHCGKREKITHSDETVRY

SEQ ID NO: 5 - DNA, 25 bp, Artificial Sequence
  synthetic nucleotide sequence
gcgcacatgaggatcacccatgtgc

SEQ ID NO: 6 - DNA, 14 bp, Artificial Sequence
  synthetic nucleotide sequence
ccacagtcactggg

SEQ ID NO: 7 - DNA, 31 bp, Artificial Sequence
  synthetic nucleotide sequence
aacataaggagtttatatggaaacccttatg

SEQ ID NO: 8 - DNA, 19 bp, Artificial Sequence
  synthetic nucleotide sequence
ctgaatgcctgcgagcatc

SEQ ID NO: 9 - DNA, 67 bp, Artificial Sequence
  synthetic nucleotide sequence
gggagcacatgaggatcacccatgtgccacgagcgacatgaggatcacccatgtcgctcgtgttccc

SEQ ID NO: 10 - DNA, 56 bp, Artificial Sequence
  synthetic nucleotide sequence
gggagcacatgaggatcacccatgtgcgactcccacagtcactggggagtcttccc

SEQ ID NO: 11 - DNA, 79 bp, Artificial Sequence
  synthetic nucleotide sequence
gggagctaaggagtttatatggaaacccttagcctgctgcgtaaggagtttatatggaaacccttacgcagcagttccc

SEQ ID NO: 12 - DNA, 69 bp, Artificial Sequence
  synthetic nucleotide sequence
gggagcacatgaggatcacccatgtgccacgagtaaggagtttatatggaaacccttactcgtgttccc

SEQ ID NO: 13 - DNA, 86 bp, Artificial Sequence
  synthetic nucleotide sequence
gtttaagagctatgctggaaacagcatagcaagtttaaataaggctagtccgttatcaacttgaaaaagtggcaccgagtcggtgc

SEQ ID NO: 14 - DNA, 7 bp, Artificial Sequence
  synthetic nucleotide sequence
ttttttt

SEQ ID NO: 15 - DNA, 20 bp, Artificial Sequence
  synthetic nucleotide sequence
tttttttgttttttatgtct

SEQ ID NO: 16 - protein, 266 aa, Homo sapiens [sequence omitted]

SEQ ID NO: 17 - protein, 49 aa, Homo sapiens
TRFVKWPRYIRLQRQRAILYKRLKVPPAINQFTQALDRQTATQLLKLAH

SEQ ID NO: 18 - protein, 61 aa, Homo sapiens
KYRPETKQEKKQRLLARAEKKAAGKGDVPTKRPPVLRAGVNTVTTLVENKKAQLVVIAHDV

SEQ ID NO: 19 - DNA, 21 bp, Artificial Sequence
  synthetic nucleotide sequence
gtacgttctctatcactgata

SEQ ID NO: 20 - DNA, 27 bp, Artificial Sequence
  synthetic nucleotide sequence
gcatacttctgcctgctggggagcctg

SEQ ID NO: 21 - DNA, 20 bp, Artificial Sequence
  synthetic nucleotide sequence
gaatagctcagaggccgagg

SEQ ID NO: 22 - DNA, 21 bp, Artificial Sequence
  synthetic nucleotide sequence
ggctaggaacgcgtctctctg

SEQ ID NO: 23 - DNA, 23 bp, Artificial Sequence
  synthetic nucleotide sequence
gcctgaagacaggtgggaagcgc

SEQ ID NO: 24 - DNA, 21 bp, Artificial Sequence
  synthetic nucleotide sequence
gagccggacaggacctcccag

SEQ ID NO: 25 - DNA, 21 bp, Artificial Sequence
  synthetic nucleotide sequence
gcgggtggtcggtagtgagtc

SEQ ID NO: 26 - DNA, 23 bp, Artificial Sequence
  synthetic nucleotide sequence
ggaccctgctgtttgcgggtggt

SEQ ID NO: 27 - DNA, 23 bp, Artificial Sequence
  synthetic nucleotide sequence
gcagacgcgaggaaggagggcgc

SEQ ID NO: 28 - DNA, 20 bp, Artificial Sequence
  synthetic nucleotide sequence
gcaagtcactccccttccct

SEQ ID NO: 29 - DNA, 24 bp, Artificial Sequence
  synthetic nucleotide sequence
gaattccatccactttagcaagga

SEQ ID NO: 30 - DNA, 23 bp, Artificial Sequence
  synthetic nucleotide sequence
gcccgcgcttcccacctgtcttc

SEQ ID NO: 31 - DNA, 25 bp, Artificial Sequence
  synthetic nucleotide sequence
gcctctgggaggtcctgtccggctc

SEQ ID NO: 32 - DNA, 20 bp, Artificial Sequence
  synthetic nucleotide sequence
acttttctctatcactgata

SEQ ID NO: 33 - DNA, 20 bp, Artificial Sequence
  synthetic nucleotide sequence
ttgatatttaagttaataaa

SEQ ID NO: 34 - DNA, 20 bp, Artificial Sequence
  synthetic nucleotide sequence
atatatagagttagagttta

SEQ ID NO: 35 - DNA, 20 bp, Artificial Sequence
  synthetic nucleotide sequence
catcgcatcaacttaaacat

SEQ ID NO: 36 - DNA, 20 bp, Artificial Sequence
  synthetic nucleotide sequence
aagacggaaaaaagtagcta

SEQ ID NO: 37 - DNA, 20 bp, Artificial Sequence
  synthetic nucleotide sequence
ttagctacttttttccgtct

SEQ ID NO: 38 - DNA, 20 bp, Artificial Sequence
  synthetic nucleotide sequence
tgaattgaatgctttgagtt

SEQ ID NO: 39 - DNA, 20 bp, Artificial Sequence
  synthetic nucleotide sequence
ttttaatctggcttacagat

SEQ ID NO: 40 - DNA, 20 bp, Artificial Sequence
  synthetic nucleotide sequence
tttaaagtgattaaaatatg

SEQ ID NO: 41 - DNA, 20 bp, Artificial Sequence
  synthetic nucleotide sequence
ttaatcactttaaaataaaa

SEQ ID NO: 42 - DNA, 20 bp, Artificial Sequence
  synthetic nucleotide sequence
tgagagaatgagagttttgt

SEQ ID NO: 43 - DNA, 20 bp, Artificial Sequence
  synthetic nucleotide sequence
atagcaccgtaccataccct

SEQ ID NO: 44 - DNA, 20 bp, Artificial Sequence
  synthetic nucleotide sequence
atttcgagtttccaagggta

SEQ ID NO: 45 - DNA, 20 bp, Artificial Sequence
  synthetic nucleotide sequence
aagcaaaggaggggaagcac

SEQ ID NO: 46 - DNA, 20 bp, Artificial Sequence
  synthetic nucleotide sequence
gtgctacgaagtggtgtctg

SEQ ID NO: 47 - DNA, 20 bp, Artificial Sequence
  synthetic nucleotide sequence
cgcagggaggtctgggtgtg

SEQ ID NO: 48 - DNA, 20 bp, Artificial Sequence
  synthetic nucleotide sequence
acccagacctccctgcgagc

SEQ ID NO: 49 - DNA, 20 bp, Artificial Sequence
  synthetic nucleotide sequence
ggagcaacgggcaaccgttt

SEQ ID NO: 50 - DNA, 21 bp, Artificial Sequence
  synthetic nucleotide sequence
ttgataacggactagccttat

SEQ ID NO: 51 - DNA, 119 bp, Artificial Sequence
  synthetic nucleotide sequence
acttttctctatcactgatagttttagagctagaaatagcaagttaaaataaggctagtccgttatcaacttgaaaaagtggcaccgagtcggtggtgctttttttgttttttatgtct

SEQ ID NO: 52 - DNA, 141 bp, Artificial Sequence
  synthetic nucleotide sequence
acttttctctatcactgatagttttagagctagaaatagcaagttaaaataaggctagtccgttatcaacttgaaaaagtggcaccgagtcggtgcgcgcacatgaggatcacccatgtgctttttttgttttttatgtct

SEQ ID NO: 53 - DNA, 114 bp, Artificial Sequence
  synthetic nucleotide sequence
gtacgttctctatcactgatagtttaagagctatgctggaaacagcatagcaagtttaaataaggctagtccgttatcaacttgaaaaagtggcaccgagtcggtgcttttttt

SEQ ID NO: 54 - DNA, 152 bp, Artificial Sequence
  synthetic nucleotide sequence
gtacgttctctatcactgatagtttaagagctatgctggaaacagcatagcaagtttaaataaggctagtccgttatcaacttgaaaaagtggcaccgagtcggtgcgcgcacatgaggatcacccatgtgctttttttgttttttatgtct

SEQ ID NO: 55 - DNA, 143 bp, Artificial Sequence
  synthetic nucleotide sequence
acttttctctatcactgatagttttagagctagaaatagcaagttaaaataaggctagtccgttatcaacttgaaaaagtggcaccgagtcggtggtgctttttttgttttttatgtctctgcagagttcggtaccagctttt